SEQUENCE LISTING (54 sequences)

SEQ ID NO: 1 - DNA, 1455 bp, Dehalococcoides mccartyi
atgaaatcgcattccacaatgagtcgccgagattttatgaaatccataggtttaggttccgcagccatagctagcatgggtgcaaccgctccctttttccatgatctagacgaaatgacaggcataggtgctgccgaaacttttaattccacgacatctatgcaaaaacgtccttggtgggttaaagaagttgacattccaacagtagaaatcgacttgaaactacgcacgccttacgccggccctacgccattagctggaacattagcctcaatatatgttaccaaagaagagactgctgctatcctggcttcccaaaagaacaatgccattgaaggagccaaaaacaaccggccaggttttacattgcgtgatcaaattggagcctgggcctcgttagatagaggacaaacgggatatgtgaaatatccgcctgaaggttttcgaacaattaaagtcacccatgaaactttaggtgtaccgaagtgggaaggctccgaaacggaaaacgcattcatgattcgaactttcctgaggcaatttggtgcaggggcgattggctatgcaagagtggatgacgacagcgtcggaccacgtaaaccccttttcaatacacatgtgagattggaaaacaacgcagattataagtacgattctaatggcgtatttgtcatgccagaaaaatgcaagtatgccattattatgtatgacagaagtccccgagatcctaacaactatcgtcgtactgtgaatagccctcaagcttttgtatcaaacatggaaaaatgtgagtatggtcataagcttcaaaacttcctttggggcttaggctaccagtcttattggtttgaagacggtacaactagtaagtttactgggaccccgactaatgtttggggtattctctcaggtgtaggagagtataaccgaattcacaatgctgtttcacaaccagaaggcgagagcggcaattttgcaagtattctctttaccgatttacctttgcccacaactaaacctatagactttggtgccttggaattctgtaaaacttgtgggatatgtgccgacgtttgcccagccggagcaattcctacagtagaagaatatcgagagccaacttgggatcgagcaactggtccctggagtgcttccaatgaccataaaggatatcctaataaatccattgaatgcgtaaaatggtatttttcctatgcaattacagcgtacgccccttcatctcgcccagttggtgtgtgtcgtcgatgtgccagccattgtgtctttagtaaagatcatgaagcttggattcatgaagtagttaagggtgtagtttccactacccctgtgatgaacagcttctttactaaaatggatatgctatccggttacagtgacgtcatctcagatgaaggcagagctgaatattggcaccagtacctgcccgctgtttaa

SEQ ID NO: 2 - protein, 484 aa, Dehalococcoides mccartyi
MKSHSTMSRRDFMKSIGLGSAAIASMGATAPFFHDLDEMTGIGAAETFNSTTSMQKRPWWVKEVDIPTVEIDLKLRTPYAGPTPLAGTLASIYVTKEETAAILASQKNNAIEGAKNNRPGFTLRDQIGAWASLDRGQTGYVKYPPEGFRTIKVTHETLGVPKWEGSETENAFMIRTFLRQFGAGAIGYARVDDDSVGPRKPLFNTHVRLENNADYKYDSNGVFVMPEKCKYAIIMYDRSPRDPNNYRRTVNSPQAFVSNMEKCEYGHKLQNFLWGLGYQSYWFEDGTTSKFTGTPTNVWGILSGVGEYNRIHNAVSQPEGESGNFASILFTDLPLPTTKPIDFGALEFCKTCGICADVCPAGAIPTVEEYREPTWDRATGPWSASNDHKGYPNKSIECVKWYFSYAITAYAPSSRPVGVCRRCASHCVFSKDHEAWIHEVVKGVVSTTPVMNSFFTKMDMLSGYSDVISDEGRAEYWHQYLPAV

SEQ ID NO: 3 - DNA, 1455 bp, Dehalococcoides mccartyi
atgaaatcgcattccacaatgagtcgccgagattttatgaaatccataggtttaggttccgcagccatagctagcatgggtgcaaccgctccctttttccatgatctagacgaaatgacaggcataggtgctgccgaaacttttaattccacgacatctatgcaaaaacgtccttggtgggttaaagaagttgacattccaacagtagaaatcgacttgaaactacgcacgccttacgccggccctacgccatcagctggaacattagcctcaatatatgttaccaaagaagagactgctgctatcctggcttcccaaaagaacaatgccattgaaggagccaaaaacaaccggccaggttttacattgcgtgatcaaattggagcctgggcctcgttagatagaggacaaacgggatatgtgaaatatccgcctgaaggttttcgaacaattaaagtcacccatgaaactttaggtgtaccgaagtgggaaggctccgaaacggaaaacgcattcatgattcgaactttcctgaggcaatttggtgcaggggcgattggctatgcaagagtggatgacgacagcgtcggaccacgtaaaccccttttcaatacacatgtgagattggaaaacaacgcagattataagtacgattctaatggcgtatttgtcatgccagaaaaatgcaagtatgccattattatgtatgacagaagtccccgagatcctaacaactatcgtcgtactgtgaatagccctcaagcttttgtatcaaacatggaaaaatgtgagtatggtcataagcttcaaaacttcctttggggcttaggctaccagtcttattggtttgaagacggtacaactagtaagtttactgggaccccgactaatgtttggggtattctctcaggtgtaggagagtataaccgaattcacaatgctgtttcacaaccagaaggcgagagcggcaattttgcaagtattctctttaccgatttacctttgcccacaactaaacctatagactttggtgccttggaattctgtaaaacttgtgggatatgtgccgacgtttgcccagccggagcaattcctacagtagaagaatatcgagagccaacttgggatcgagcaactggtccctggagtgcttccaatgaccataaaggatatcctaataaatccattgaatgcgtaaaatggtatttttcctatgcaattacagcgtacgccccttcatctcgcccagttggtgtgtgtcgtcgatgtgccagccattgtgtctttagtaaagatcatgaagcttggattcatgaagtagttaagggtgtagtttccactacccctgtgatgaacagcttctttactaaaatggatatgctatccggttacagtgacgtcatctcagatgaaggcagagctgaatattggcaccagtacctgcccgctgtttaa

SEQ ID NO: 4 - protein, 484 aa, Dehalococcoides mccartyi
MKSHSTMSRRDFMKSIGLGSAAIASMGATAPFFHDLDEMTGIGAAETFNSTTSMQKRPWWVKEVDIPTVEIDLKLRTPYAGPTPSAGTLASIYVTKEETAAILASQKNNAIEGAKNNRPGFTLRDQIGAWASLDRGQTGYVKYPPEGFRTIKVTHETLGVPKWEGSETENAFMIRTFLRQFGAGAIGYARVDDDSVGPRKPLFNTHVRLENNADYKYDSNGVFVMPEKCKYAIIMYDRSPRDPNNYRRTVNSPQAFVSNMEKCEYGHKLQNFLWGLGYQSYWFEDGTTSKFTGTPTNVWGILSGVGEYNRIHNAVSQPEGESGNFASILFTDLPLPTTKPIDFGALEFCKTCGICADVCPAGAIPTVEEYREPTWDRATGPWSASNDHKGYPNKSIECVKWYFSYAITAYAPSSRPVGVCRRCASHCVFSKDHEAWIHEVVKGVVSTTPVMNSFFTKMDMLSGYSDVISDEGRAEYWHQYLPAV

SEQ ID NO: 5 - DNA, 219 bp, Dehalococcoides mccartyi
atgaattttaatattgactttaaatggtatcaatggctttttggagtgatttctcttattctggcatctttcttgactcatgaagtatttgctaccctagcagaatctcagcctggcactgtcaaggtactttccttgcttataggaatcccattgattatcttcctgtatctgacctttggtttaaggtcggccttaaaaaaacacaagtctaactaa

SEQ ID NO: 6 - protein, 72 aa, Dehalococcoides mccartyi
MNFNIDFKWYQWLFGVISLILASFLTHEVFATLAESQPGTVKVLSLLIGIPLIIFLYLTFGLRSALKKHKSN

SEQ ID NO: 7 - DNA, 219 bp, Dehalococcoides mccartyi
atgaattttaatattgactttaaatggtatgaatggctttttggagtgatttctcttattctggcatctttcttgactcatgaagtatttgctaccctagcagaatctcagcctggcactgtcaaggtactttccttgcttataggaatcccattgattatcttcctgtatctgacctttggtttaaggtcggccttaaaaaaacacaagtctaactaa

SEQ ID NO: 8 - protein, 72 aa, Dehalococcoides mccartyi
MNFNIDFKWYEWLFGVISLILASFLTHEVFATLAESQPGTVKVLSLLIGIPLIIFLYLTFGLRSALKKHKSN

SEQ ID NO: 9 - DNA, 1781 bp, Dehalococcoides mccartyi
gtgtcttgtcagcctccctgtgttcattctagtaaacaagggttacagagtcaaggaaccaacaaaaatcaaaacataaaagaggtaatatgaaatcgcattccacaatgagtcgccgagattttatgaaatccataggtttaggttccgcagccatagctagcatgggtgcaaccgctccctttttccatgatctagacgaaatgacaggcataggtgctgccgaaacttttaattccacgacatctatgcaaaaacgtccttggtgggttaaagaagttgacattccaacagtagaaatcgacttgaaactacgcacgccttacgccggccctacgccattagctggaacattagcctcaatatatgttaccaaagaagagactgctgctatcctggcttcccaaaagaacaatgccattgaaggagccaaaaacaaccggccaggttttacattgcgtgatcaaattggagcctgggcctcgttagatagaggacaaacgggatatgtgaaatatccgcctgaaggttttcgaacaattaaagtcacccatgaaactttaggtgtaccgaagtgggaaggctccgaaacggaaaacgcattcatgattcgaactttcctgaggcaatttggtgcaggggcgattggctatgcaagagtggatgacgacagcgtcggaccacgtaaaccccttttcaatacacatgtgagattggaaaacaacgcagattataagtacgattctaatggcgtatttgtcatgccagaaaaatgcaagtatgccattattatgtatgacagaagtccccgagatcctaacaactatcgtcgtactgtgaatagccctcaagcttttgtatcaaacatggaaaaatgtgagtatggtcataagcttcaaaacttcctttggggcttaggctaccagtcttattggtttgaagacggtacaactagtaagtttactgggaccccgactaatgtttggggtattctctcaggtgtaggagagtataaccgaattcacaatgctgtttcacaaccagaaggcgagagcggcaattttgcaagtattctctttaccgatttacctttgcccacaactaaacctatagactttggtgccttggaattctgtaaaacttgtgggatatgtgccgacgtttgcccagccggagcaattcctacagtagaagaatatcgagagccaacttgggatcgagcaactggtccctggagtgcttccaatgaccataaaggatatcctaataaatccattgaatgcgtaaaatggtatttttcctatgcaattacagcgtacgccccttcatctcgcccagttggtgtgtgtcgtcgatgtgccagccattgtgtctttagtaaagatcatgaagcttggattcatgaagtagttaagggtgtagtttccactacccctgtgatgaacagcttctttactaaaatggatatgctatccggttacagtgacgtcatctcagatgaaggcagagctgaatattggcaccagtacctgcccgctgtttaaaatgagagaggaaaaaacatgaattttaatattgactttaaatggtatcaatggctttttggagtgatttctcttattctggcatctttcttgactcatgaagtatttgctaccctagcagaatctcagcctggcactgtcaaggtactttccttgcttataggaatcccattgattatcttcctgtatctgacctttggtttaaggtcggccttaaaaaaacacaagtctaactaa

SEQ ID NO: 10 - DNA, 1781 bp, Dehalococcoides mccartyi
gtgtcttgtcagcctccctgtgttcattctagtaaacaagggttacagagtcaaggaaccaacaaaaatcaaaacataaaagaggtaatatgaaatcgcattccacaatgagtcgccgagattttatgaaatccataggtttaggttccgcagccatagctagcatgggtgcaaccgctccctttttccatgatctagacgaaatgacaggcataggtgctgccgaaacttttaattccacgacatctatgcaaaaacgtccttggtgggttaaagaagttgacattccaacagtagaaatcgacttgaaactacgcacgccttacgccggccctacgccatcagctggaacattagcctcaatatatgttaccaaagaagagactgctgctatcctggcttcccaaaagaacaatgccattgaaggagccaaaaacaaccggccaggttttacattgcgtgatcaaattggagcctgggcctcgttagatagaggacaaacgggatatgtgaaatatccgcctgaaggttttcgaacaattaaagtcacccatgaaactttaggtgtaccgaagtgggaaggctccgaaacggaaaacgcattcatgattcgaactttcctgaggcaatttggtgcaggggcgattggctatgcaagagtggatgacgacagcgtcggaccacgtaaaccccttttcaatacacatgtgagattggaaaacaacgcagattataagtacgattctaatggcgtatttgtcatgccagaaaaatgcaagtatgccattattatgtatgacagaagtccccgagatcctaacaactatcgtcgtactgtgaatagccctcaagcttttgtatcaaacatggaaaaatgtgagtatggtcataagcttcaaaacttcctttggggcttaggctaccagtcttattggtttgaagacggtacaactagtaagtttactgggaccccgactaatgtttggggtattctctcaggtgtaggagagtataaccgaattcacaatgctgtttcacaaccagaaggcgagagcggcaattttgcaagtattctctttaccgatttacctttgcccacaactaaacctatagactttggtgccttggaattctgtaaaacttgtgggatatgtgccgacgtttgcccagccggagcaattcctacagtagaagaatatcgagagccaacttgggatcgagcaactggtccctggagtgcttccaatgaccataaaggatatcctaataaatccattgaatgcgtaaaatggtatttttcctatgcaattacagcgtacgccccttcatctcgcccagttggtgtgtgtcgtcgatgtgccagccattgtgtctttagtaaagatcatgaagcttggattcatgaagtagttaagggtgtagtttccactacccctgtgatgaacagcttctttactaaaatggatatgctatccggttacagtgacgtcatctcagatgaaggcagagctgaatattggcaccagtacctgcccgctgtttaaaatgagagaggaaaaaacatgaattttaatattgactttaaatggtatgaatggctttttggagtgatttctcttattctggcatctttcttgactcatgaagtatttgctaccctagcagaatctcagcctggcactgtcaaggtactttccttgcttataggaatcccattgattatcttcctgtatctgacctttggtttaaggtcggccttaaaaaaacacaagtctaactaa

SEQ ID NO: 11 - DNA, 21 bp, Artificial Sequence
  Description of Artificial Sequence Synthetic primer
shmgbmgwgatttyatgaarr

SEQ ID NO: 12 - DNA, 19 bp, Artificial Sequence
  Description of Artificial Sequence Synthetic primer
chadhagccaytcrtacca

SEQ ID NO: 13 - DNA, 17 bp, Artificial Sequence
  Description of Artificial Sequence Synthetic primer
gtaaaacgacggccagt

SEQ ID NO: 14 - DNA, 19 bp, Artificial Sequence
  Description of Artificial Sequence Synthetic primer
ggaaacagctatgaccatg

SEQ ID NO: 15 - DNA, 21 bp, Artificial Sequence
  Description of Artificial Sequence Synthetic primer
gctcctggcagagccgtcagt

SEQ ID NO: 16 - DNA, 24 bp, Artificial Sequence
  Description of Artificial Sequence Synthetic primer
tttaaacagcgggcaggtactggt

SEQ ID NO: 17 - DNA, 24 bp, Artificial Sequence
  Description of Artificial Sequence Synthetic primer
ttgcgtgatcaaattggagcctgg

SEQ ID NO: 18 - DNA, 24 bp, Artificial Sequence
  Description of Artificial Sequence Synthetic primer
cgatgtgccagccattgtgtcttt

SEQ ID NO: 19 - DNA, 21 bp, Artificial Sequence
  Description of Artificial Sequence Synthetic primer
ctggagctaatccccaaagct

SEQ ID NO: 20 - DNA, 26 bp, Artificial Sequence
  Description of Artificial Sequence Synthetic primer
acgtcatctcagatgaaggcagagct

SEQ ID NO: 21 - DNA, 22 bp, Artificial Sequence
  Description of Artificial Sequence Synthetic primer
tgagtcgccgagattttatgaa

SEQ ID NO: 22 - DNA, 20 bp, Artificial Sequence
  Description of Artificial Sequence Synthetic primer
tgcacccatgctagctatgg

SEQ ID NO: 23 - DNA, 20 bp, Artificial Sequence
  Description of Artificial Sequence Synthetic primer
tccataggtttaggttccgc

SEQ ID NO: 24 - DNA, 16 bp, Artificial Sequence
  Description of Artificial Sequence Synthetic primer
cggccctacgccatca

SEQ ID NO: 25 - DNA, 24 bp, Artificial Sequence
  Description of Artificial Sequence Synthetic primer
gcagcagtctcttctttggtaaca

SEQ ID NO: 26 - DNA, 19 bp, Artificial Sequence
  Description of Artificial Sequence Synthetic primer
ctggaacattagcctcaat

SEQ ID NO: 27 - DNA, 22 bp, Artificial Sequence
  Description of Artificial Sequence Synthetic primer
gccaggttttacattgcgtgat

SEQ ID NO: 28 - DNA, 23 bp, Artificial Sequence
  Description of Artificial Sequence Synthetic primer
tcccgtttgtcctctatctaacg

SEQ ID NO: 29 - DNA, 16 bp, Artificial Sequence
  Description of Artificial Sequence Synthetic primer
aattggagcctgggcc

SEQ ID NO: 30 - DNA, 18 bp, Artificial Sequence
  Description of Artificial Sequence Synthetic primer
ccgcctgaaggttttcga

SEQ ID NO: 31 - DNA, 20 bp, Artificial Sequence
  Description of Artificial Sequence Synthetic primer
gccttcccacttcggtacac

SEQ ID NO: 32 - DNA, 18 bp, Artificial Sequence
  Description of Artificial Sequence Synthetic primer
taaagtcacccatgaaac

SEQ ID NO: 33 - DNA, 19 bp, Artificial Sequence
  Description of Artificial Sequence Synthetic primer
aaggctccgaaacggaaaa

SEQ ID NO: 34 - DNA, 17 bp, Artificial Sequence
  Description of Artificial Sequence Synthetic primer
cgcccctgcaccaaatt

SEQ ID NO: 35 - DNA, 23 bp, Artificial Sequence
  Description of Artificial Sequence Synthetic primer
attcatgattcgaactttcctga

SEQ ID NO: 36 - DNA, 22 bp, Artificial Sequence
  Description of Artificial Sequence Synthetic primer
ccaggttttacattgcgtgatc

SEQ ID NO: 37 - DNA, 23 bp, Artificial Sequence
  Description of Artificial Sequence Synthetic primer
tcccgtttgtcctctatctaacg

SEQ ID NO: 38 - DNA, 16 bp, Artificial Sequence
  Description of Artificial Sequence Synthetic primer
aattggagcctgggcc

SEQ ID NO: 39 - DNA, 18 bp, Artificial Sequence
  Description of Artificial Sequence Synthetic primer
ccgcctgaaggttttcga

SEQ ID NO: 40 - DNA, 23 bp, Artificial Sequence
  Description of Artificial Sequence Synthetic primer
cttcccacttcggtacacctaaa

SEQ ID NO: 41 - DNA, 20 bp, Artificial Sequence
  Description of Artificial Sequence Synthetic primer
aattaaagtcacccatgaaa

SEQ ID NO: 42 - DNA, 22 bp, Artificial Sequence
  Description of Artificial Sequence Synthetic primer
tgagtcgccgagattttatgaa

SEQ ID NO: 43 - DNA, 20 bp, Artificial Sequence
  Description of Artificial Sequence Synthetic primer
tgcacccatgctagctatgg

SEQ ID NO: 44 - DNA, 20 bp, Artificial Sequence
  Description of Artificial Sequence Synthetic primer
tccataggtttaggttccgc

SEQ ID NO: 45 - DNA, 16 bp, Artificial Sequence
  Description of Artificial Sequence Synthetic primer
ccggccctacgccatt

SEQ ID NO: 46 - DNA, 24 bp, Artificial Sequence
  Description of Artificial Sequence Synthetic primer
gcagcagtctcttctttggtaaca

SEQ ID NO: 47 - DNA, 19 bp, Artificial Sequence
  Description of Artificial Sequence Synthetic primer
ctggaacattagcctcaat

SEQ ID NO: 48 - DNA, 19 bp, Artificial Sequence
  Description of Artificial Sequence Synthetic primer
aaggctccgaaacggaaaa

SEQ ID NO: 49 - DNA, 17 bp, Artificial Sequence
  Description of Artificial Sequence Synthetic primer
cgcccctgcaccaaatt

SEQ ID NO: 50 - DNA, 23 bp, Artificial Sequence
  Description of Artificial Sequence Synthetic primer
attcatgattcgaactttcctga

SEQ ID NO: 51 - DNA, 20 bp, Artificial Sequence
  Description of Artificial Sequence Synthetic primer
agagtttgatcctggctcag

SEQ ID NO: 52 - DNA, 20 bp, Artificial Sequence
  Description of Artificial Sequence Synthetic primer
aaggaggtgatccagccgca

SEQ ID NO: 53 - DNA, 18 bp, Artificial Sequence
  Description of Artificial Sequence Synthetic primer
caacttcatgcaggcggg

SEQ ID NO: 54 - DNA, 25 bp, Artificial Sequence
  Description of Artificial Sequence Synthetic probe
tcctcagttcggattgcaggctgaa

(19) United States
(12) Patent Application Publication
(10) Pub. No.: US 2014/0072965 A1
(43) Pub. Date: Mar. 13, 2014

(54) 1, 2-Dichloropropane-to-Propene Reductive Dehalogenase Genes

(71) Applicants: Elizabeth Padilla-Crespo, Knoxville, TN (US); Frank E. Loeffler, Knoxville, TN (US); Kirsti M. Ritalahti, Knoxville, TN (US)

(72) Inventors: Elizabeth Padilla-Crespo, Knoxville, TN (US); Frank E. Loeffler, Knoxville, TN (US); Kirsti M. Ritalahti, Knoxville, TN (US)

(73) Assignee: UNIVERSITY OF TENNESSEE RESEARCH FOUNDATION, Knoxville, TN (US)

(21) Appl. No.: 13/607,505

(22) Filed: Sep. 7, 2012

(51) Int. Cl. C12N 15/55; C12Q 1/68; C12N 5/10; G01N 21/00; C12N 1/19; C12N 1/21; C12N 15/63; C07H 21/04

(52) U.S. Cl. 435/6.11; 536/23.2; 435/320.1; 435/6.12; 536/24.33; 536/24.32; 435/254.2; 435/252.3; 435/348; 435/252.33; 435/252.31; 435/254.21

(57) ABSTRACT

The invention is directed to novel reductive dehalogenase genes encoding for reductive dehalogenases, which are capable of dehalogenating halogenated organic compounds and may be useful for environmental assessment and monitoring, and in the bioremediation of pollutants. In particular, the invention provides isolated polynucleotides of novel reductive dehalogenase genes dcpA and dcpB and fragments thereof as well as isolated polypeptides encoding the DcpA and DcpB proteins or fragments thereof. The invention is also directed to methods of identifying and/or quantifying dechlorinating bacterial organisms or polynucleotides encoding a reductive dehalogenase, such as the dcpA or dcpB polynucleotides or fragments thereof, in a sample.

### STATEMENT OF GOVERNMENT SUPPORT

This invention was made with government support under contract W912HQ-10-C-0062 (project ER-1586) awarded by the Strategic Environmental Research and Development Program (SERDP) and under fellowships from the National Science Foundation. The Government has certain rights in this invention.

### REFERENCE TO SEQUENCE LISTING

The instant application contains a Sequence Listing which has been submitted in ASCII format via EFS-Web and is hereby incorporated by reference in its entirety. Said ASCII copy, created on Sep. 7, 2012, is named 21597_CRF_sequencelisting.txt and is 29,668 bytes in size.

### FIELD OF THE INVENTION

The invention relates to novel 1,2-dichloropropane-to-propene reductive dehalogenase genes and proteins that have been isolated from bacteria. The invention also relates to methods of detecting, quantifying and characterizing populations of bacteria possessing the novel 1,2-dichloropropane-to-propene reductive dehalogenase genes of the invention and methods of detecting and quantifying the novel 1,2-dichloropropane-to-propene reductive dehalogenase genes or portions thereof.

### BACKGROUND OF THE INVENTION

1,2-dichloropropane has been used in a variety of applications including as an industrial solvent, a lead scavenger in gasoline, and a fumigant to prevent root nematode damage to high value crops. (Agency for Toxic Substances and Disease Registry Report, December 1989). In addition, 1,2-dichloropropane is a precursor for the production of other chlorinated solvents and a byproduct in the production of propyleneoxide and epichlorohydrine. (Nijhuis, T. A., et al. (2006) Ind. Eng. Chem. Res. 45:3447-3459). 1,2-dichloropropane is toxic and a suspected carcinogen, and the U.S. Environmental Protection Agency (EPA) regulates its maximum concentration level (MCL) in drinking water to 5 parts per billion (ppb). (See http://water.epa.gov/drink/contaminants/index.cfm#List). Today, 1,2-dichloropropane is no longer used as a solvent or in soil fumigant applications. However, the 2010 EPA's Toxics Release Inventory reported that 93,873 pounds of 1,2-dichloropropane were disposed of or released on- or off-site. (See http://iaspub.epa.gov/triexplorer/tri_release.chemical.) But since some discharges of 1,2-dichloropropane do not fall under the EPA's reporting requirements, the EPA's reported amount of 1,2-dichloropropane release must be considered a minimum and the total amounts actually discharged into the environment are likely to exceed the EPA's estimates. A 2006 study conducted by the National Water-Quality Assessment Program (NAWQA) and lead by the United States Geological Survey (USGS) detected 1,2-dichloropropane in 1% of aquifers and private wells located proximal to agricultural areas throughout the U.S. and, in some cases, the amount of 1,2-dichloropropane was above the federal MCL of 5 ppb. (Zogorski, J., et al. (2006) US Geological Survey). Moreover, in the NAWQA aquifer study, fumigants were detected in more than 30 percent of the wells sampled in Oahu, Hi. and the Central Valley of California. (Zogorski, J., et al. (2006) US Geological Survey). These are areas where fumigant applications were common. For example, Hawaii used more than 1.8 million pounds of fumigants in the 1970's to protect pineapple crops from root-parasitic nematodes. (Pacific Biomedical Research Center. (1967) Hawaii epidemiologic studies program). Even though 1,2-dichloropropane use is now controlled and new contamination minimized, 1,2-dichloropropane is a pervasive environmental contaminant and a threat to groundwater and drinking water reservoirs.

A few bacteria have been implicated in 1,2-dichloropropane reductive dechlorination to non-toxic propene under anoxic conditions. For example, Dehalogenimonas (Dhg) strains BL-DC-8 and BL-DC-9 (Moe, W. M., et al. (2009) Int. J. Syst. Evol. Microbiol. 59:2692-2697) and Dehalococcoides (Dhc) mccartyi strains RC and KS (Lö ffler, F. E., et al. (1997) Appl. Environ. Microbiol. 63:2870-2875; Ritalahti, K. M., and F. E. Löffler (2004) Appl. Environ. Microbiol. 70:4088-4095) have been demonstrated to dechlorinate 1,2-dichloropropane under anoxic conditions. The Dhc mccartyi strains RC and KS were derived from geographical distinct locations, and are the only known Dhc strains known to dechlorinate 1,2-dichloropropane.

Reductive dechlorination reactions are catalyzed by reductive dehalogenases (RDases). To date only a few RDases have been biochemically characterized and implicated in specific dechlorination reactions in bacterial organohalide respiration. A RDase responsible for 1,2-dichloropropane-to-propene dechlorination (denominated dcpA) has not previously been identified.

### BRIEF SUMMARY OF THE INVENTION

In certain embodiments, the present invention provides novel reductive dehalogenase genes isolated from dechlorinating bacteria and encoding for reductive dehalogenase enzyme systems. The deduced amino acid sequences of the presently identified dehalogenase enzymes, as well as experimental data, indicates that they are capable of the reductive dehalogenation of halogenated substrates and in particular the reduction of 1,2-dichloropropane to propene and inorganic chloride.

In certain embodiments, the invention provides for methods of identifying and isolating bacterial target DNA from dechlorinating bacteria of interest, such as Dehalococcoides populations.

In additional embodiments, the invention provides gene primer pairs and probes useful for detection and quantification of dechlorinating bacteria using analytical techniques such as, for example and without limitation, hybridization, PCR and Quantitative Real-Time PCR (qPCR) technology. The components provided and the methods in which they are employed are useful in environmental monitoring and bioremediation processes mediated by dechlorinating bacteria.

In still another embodiment, the invention provides for isolated polynucleotides encoding a reductive dehalogenase comprising a polynucleotide sequence having at least 85% and preferably at least 90%, more preferably at least 95%, still more preferably at least 97% and still more preferably at least 99% sequence identity over the length of the entire reference sequence to a polynucleotide selected from the group consisting of SEQ ID NO: 1 and SEQ ID NO: 3. In other embodiments, the invention provides for isolated polynucleotides comprising a polynucleotide sequence having at least 85% and preferably at least 90%, more preferably at least 95%, still more preferably at least 97% and still more preferably at least 99% sequence identity over the length of the entire reference sequence to a polynucleotide selected from the group consisting of SEQ ID NO: 1, SEQ ID NO: 3, SEQ ID NO: 5, SEQ ID NO: 7, SEQ ID NO: 9, SEQ ID NO: 10, SEQ ID NO: 15, SEQ ID NO: 16, SEQ ID NO: 17, SEQ ID NO: 18, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 55, SEQ ID NO: 56, and SEQ ID NO: 57.

In other embodiments, the invention provides a recombinant expression vector comprising any one of the aforementioned isolated polynucleotides operably linked to a regulatory sequence, and a cell, or organism comprising the recombinant gene sequence. In another embodiment, the invention provides a vector comprising any one of the aforementioned isolated polynucleotides.

In still another embodiment, the invention provides isolated polynucleotides encoding an enzyme that reductively dechlorinates 1,2-dichloropropane. In a preferred embodiment, the invention provides an isolated polynucleotide encoding a reductive dehalogenase including, for example, SEQ ID NO: 1, SEQ ID NO: 3, SEQ ID NO: 9 and SEQ ID NO: 10.

In yet another embodiment, the invention provides isolated polynucleotides encoding an enzyme that reductively dechlorinates 1,2-dichloropropane wherein the polynucleotide is isolated from dechlorinating bacteria, such as for example, Dehalococcoides bacteria including, for example, Dehalococcoides (Dhc) mccartyi strains RC and KS. Such polynucleotides include, for example, SEQ ID NO: 1, SEQ ID NO: 3, SEQ ID NO: 9 and SEQ ID NO: 10.

In another embodiment, the present invention provides a method of identifying a polynucleotide encoding a reductive dehalogenase in a sample, comprising: contacting the sample with (i) a first oligonucleotide primer comprising a portion of a polynucleotide encoding an enzyme that reductively dechlorinates 1,2-dichloropropane; and (ii) a second oligonucleotide primer comprising a sequence that is complementary to a portion of a polynucleotide encoding an enzyme that reductively dechlorinates 1,2-dichloropropane; and performing PCR on the sample, wherein the presence of an amplification product indicates the presence of a polynucleotide encoding a reductive dehalogenase in the sample. In certain embodiments, the present invention provides a method of identifying a polynucleotide encoding a reductive dehalogenase in a sample, comprising: contacting the sample with (i) a first oligonucleotide primer comprising a sequence selected from the group consisting of SEQ ID NO: 15, SEQ ID NO: 17, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48 and SEQ ID NO: 55; and (ii) a second oligonucleotide primer comprising a sequence selected from the group consisting of SEQ ID NO: 16, SEQ ID NO: 22, SEQ ID NO: 25, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, and SEQ ID NO: 56; and performing PCR on the sample, wherein the presence of an amplification product indicates the presence of a polynucleotide encoding a reductive dehalogenase in the sample.

In another embodiment the invention provides a method of quantifying the amount of dechlorinating bacteria present in a sample comprising: (a) contacting the sample with (i) a probe comprising a portion of any one of the sequences selected from the group consisting of SEQ ID NO: 20, SEQ ID NO: 23, SEQ ID NO: 26, SEQ ID NO: 29, SEQ ID NO: 32, SEQ ID NO: 35, SEQ ID NO: 38, SEQ ID NO: 41, SEQ ID NO: 44, SEQ ID NO: 47, SEQ ID NO: 50, and SEQ ID NO: 57; (ii) a first primer comprising a portion of any one of the sequences selected from the group consisting of SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, and SEQ ID NO: 55; and (iii) a second primer comprising a portion of any one of the sequences selected from the group consisting of SEQ ID NO: 16, SEQ ID NO: 22, SEQ ID NO: 25, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, and SEQ ID NO: 56; and (b) performing Quantitative Real-Time PCR on the sample to enumerate the abundance of genes encoding 1,2-dichloropropane reductive dehalogenases, transcripts of the 1,2-dichloropropane encoding reductive dehalogenases, and/or dechlorinating bacteria present in the sample. In some embodiments, the probe further comprises one or more of a reporter dye and a quencher dye. For example, in some embodiments, the probe includes a 5′-reporter dye and a 3′-quencher dye.

In some embodiments, the invention provides a method of detecting the presence of a dechlorinating bacteria or a dcpA or dcpB gene in a sample comprising: (a) contacting the sample with (i) a first oligonucleotide primer comprising a portion of a polynucleotide encoding an enzyme that reductively dechlorinates 1,2-dichloropropane; and (ii) a second oligonucleotide primer comprising a sequence that is complementary to a portion of a polynucleotide encoding an enzyme that reductively dechlorinates 1,2-dichloropropane; and (b) performing PCR on the sample, wherein the presence of an amplification product confirms the presence of the dechlorinating bacteria. In another embodiment, the invention provides a method of detecting the presence of a dechlorinating bacteria or a dcpA or dcpB gene in a sample comprising:

(a) contacting the sample with (i) a first primer comprising a portion of any one of the sequences selected from the group consisting of SEQ ID NO: 15, SEQ ID NO: 17, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, and SEQ ID NO: 55; and (ii) a second primer comprising a portion of a sequence selected from the group consisting of SEQ ID NO: 16, SEQ ID NO: 22, SEQ ID NO: 25, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, and SEQ ID NO: 56; and (b) performing PCR on the sample, wherein the presence of amplification products confirms the presence of the dechlorinating bacteria.

In another embodiment, the invention provides a method for identifying a dechlorinating bacterial organism comprising the steps of (a) contacting a probe with a bacterial cell extract, the contact effecting the hybridization with a nucleic acid derived from the bacterial cell extract, wherein the probe comprises any one of the aforementioned isolated polynucleotides, or a fragment thereof, and (b) determining that the probe has hybridized to the nucleic acid derived from the bacterial cell extract.

### BRIEF DESCRIPTION OF THE DRAWINGS

FIG. 1 depicts a 1% agarose gel that has been stained with ethidium bromide. Complementary DNA (cDNA) samples prepared from RNA extracts from Dehalococcoides (Dhc) mccartyi strain RC and strain KS cultures were used as templates with the reductive dehalogenase degenerate primers RRF2 (SEQ ID NO: 11) and B1R (SEQ ID NO: 12). The Dhc mccartyi strain RC and strain KS had been grown with 1,2-dichloropropane as the electron acceptor and produced propene. Lanes 1-3 correspond to template samples from Dhc mccartyi strain RC and lanes 4-6 correspond to samples from Dhc mccartyi strain KS. Lane 1 and 4 are “no reverse transcriptase” controls; lanes 2 and 5 show PCR amplification using cDNA as template; and lanes 3 and 6 correspond to PCR positive controls using genomic DNA from the respective cultures as template DNA for the PCR reactions. The 1 Kb Plus Ladder from Invitrogen is shown in the first and last lanes of the gel.

FIG. 2 is of a 1% agarose gel that has been stained with ethidium bromide and shows the PCR amplicons obtained using the primers dcp_up120F (SEQ ID NO: 15) and dcpA-1449R (SEQ ID NO: 16). Lane 1 is a positive control that used genomic DNA from Dehalogenimonas lykanthroporepellens strain BL-DC-9 as template for the PCR reaction. A PCR product of the expected size (˜1569 bp) was also obtained using Dhc mccartyi strain RC and strain KS genomic DNA as a template (lanes 2 and 3, respectively). Lane 4 is a negative control that did not include any template DNA in the PCR reaction. The 1 Kb Plus Ladder from Invitrogen is shown in the left-most lane of the gel.

FIG. 3 is an amino acid sequence alignment of the amino acid sequence of the 1,2-dichloropropane reductive dehalogenase DcpA of Dhc mccartyi strain KS (denoted “DhcKS”; SEQ ID NO: 2) and the DcpA amino acid sequence of Dhc mccartyi strain RC (denoted “DhcRC”; SEQ ID NO: 4). The sequence alignment was prepared using the Align Sequences Protein BLAST tool (default parameters) available from the National Center for Biotechnology Information (NCBI) website at http://blast.ncbi.nlm.nih.gov/Blast.cgi?PAGE=Proteins&PROGRAM=blastp&BLAST_PROGRAMS=blastp&PAGE_TYPE=BlastSearch&BLAST_SPEC=blast2seq.

FIG. 4 is an amino acid sequence alignment of the amino acid sequence of the Dhc mccartyi KS strain DcpB protein (denoted “DhcKS”; SEQ ID NO: 6) and the amino acid sequence of the Dhc mccartyi RC strain DcpB protein (denoted “DhcRC”; SEQ ID NO: 8). The sequence alignment was prepared using the Align Sequences Protein BLAST tool (default parameters) available from the National Center for Biotechnology Information (NCBI) website at http://blast.ncbi.nlm.nih.gov/Blast.cgi?PAGE=Proteins&PROGRAM=blastp&BLAST_PROGRAMS=blastp&PAGE_TYPE=BlastSearch&BLAST_SPEC=blast2seq.

FIG. 5 is a schematic representation of the dcpA and dcpB genes that highlights features of these genes that are shared with other reductive dehalogenase genes. The conserved amino acids for the Tat signal peptide RRXFXK at the N-terminus of the DcpA protein and two iron sulfur clusters closer to the C-terminus of the DcpA protein in the form of FCXXCXXCXXXCP (or FCX₂CX₂CX₃CP) and CXXCXXXC (or CX₂CX₃C) are illustrated. Downstream of the dcpA gene is the dcpB gene that has a conserved twin motif in the form WYXW. Approximate binding sites for the degenerate primers directed to putative reductive dehalogenases, RRF2 (SEQ ID NO: 11) and B1R (SEQ ID NO: 12), as well as some dcpA specific primers described herein are indicated.

FIG. 6 is a collection of the data acquired from the Quantitative Real-Time PCR assay experiments described in Example 6 herein. FIG. 6A (left panel) shows the melting curve analyses performed with the SYBR Green assay with primers dcpA-1257F (SEQ ID NO: 18) and dcpA-1449R (SEQ ID NO: 16) using genomic DNA of Dehalococcoides (Dhc) mccartyi strain RC and strain KS and Dehalogenimonas lykanthroporepellens strain BL-DC-9 as template. Also shown in FIG. 6A (top-right panel) are examples of TaqMan amplification curves obtained for a 10-fold dilution series of template DNA, with the dcpA gene-targeted primers dcpA-1257F (SEQ ID NO: 18) and dcpA-1449R (SEQ ID NO: 16) and the dcpA-1426 Probe (SEQ ID NO: 20), spanning a range of 10⁸ to 10⁰ dcpA copies per reaction. Additionally, dcpA TaqMan PCR amplification products were analyzed by electrophoresis to confirm assay specificity (FIG. 6A; bottom-right panel). Lanes 1-3 are from reactions that used 10 ng of template DNA from Dhc mccartyi strain RC, Dhc mccartyi strain KS, and Dehalogenimonas (Dhg) strain BL-DC-9, respectively. Lane 4 is a no template control and lane 5 corresponds to a reaction that had 2 ng of plasmid DNA carrying the dcpA gene fragment. The left most lane on the gel includes a 1 kb Plus DNA Ladder (Invitrogen). FIG. 6B shows standard curves for dcpA using 10-fold serial dilutions of plasmids with the inserts of interest. TaqMan Quantitative Real-Time PCR assays were performed using partial plasmid DNA carrying the dcpA gene fragment of Dehalococcoides strain KS (gray squares) and Dehalogenimonas (Dhg) strain BL-DC-9 (black diamonds) as the template. Also included is the standard curve for Dehalococcoides 16S rRNA gene quantification (white triangles). The standard curve shown has a dynamic range of 10⁹ to 10⁰ gene copies per μL of template DNA.

FIG. 7 is a 1% agarose gel of dcpA PCR products that has been stained with ethidium bromide. PCR was performed with the specific primers dcpA-360F (SEQ ID NO: 17)/dcpA-1449R (SEQ ID NO: 16). Lanes 2-4 correspond to direct PCR performed on 10 ng of genomic DNA of Dehalococcoides (Dhc) mccartyi strain RC and strain KS and the Dehalogenimonas lykanthroporepellens (Dhg) strain BL-DC-9, respectively, in 20-μL total volume reactions. Lane 1 is a PCR control with no template DNA. The marker used on the left is the 1 kb Plus DNA Ladder (Invitrogen). For all samples five μl of PCR product were mixed with 1 μL of 6× loading dye and resolved on 1% (wt/vol) agarose gels and stained with ethidium bromide (1 μg/mL).

FIG. 8 is a bar graph quantifying the Dehalococcoides (Dhc) 16S rRNA and dcpA genes using Quantitative Real-Time PCR as discussed in Example 9 herein. Quantitative Real-Time PCR was performed on duplicate Dehalococcoides (Dhc) mccartyi RC and KS cultures that had reduced the 1,2-dichloropropane in the culture media to propene. The dcpA gene-targeted primers dcpA-1257F (SEQ ID NO: 18) and dcpA-1449R (SEQ ID NO: 16) and the dcpA-1426Probe (SEQ ID NO: 20) was used to quantify the abundance of the dcpA gene. The group-specific 16S rRNA gene-targeted primers Dhc 1200F (SEQ ID NO: 19) and Dhc 1271R (SEQ ID NO: 53) and the probe Dhc-1240Probe (SEQ ID NO: 54) were used to quantify the 16S rRNA gene. Samples were run in triplicate and represent two independent DNA extractions. Error bars are provided and, if not visible in the Figure, are too small to be seen.

FIG. 9 is bar graph representing experimental data that analyzes the gene expression level of dcpA in Dehalococcoides (Dhc) mccartyi strains RC and KS grown in the presence or absence of 1,2-dichloropropane as indicated in the figure legend. dcpA transcript levels were normalized to rpoB transcript levels or to dcpA gene copy number. Samples were run in triplicate on the ABI 7500 fast Real-Time PCR system (Applied Biosystems) and final values represent the average of at least three biological replicate cultures. Error bars depict standard error.

### DETAILED DESCRIPTION OF THE INVENTION

The present invention is directed to novel reductive dehalogenase genes encoding for reductive dehalogenases which are capable of dehalogenating organic compounds. The genes and proteins they encode may be useful in the bioremediation of pollutants. In particular embodiments, the invention provides the complete sequence of novel reductive dehalogenase genes (dcpA and dcpB), having the polynucleotide sequence of SEQ ID NO: 1, SEQ ID NO: 3, SEQ ID NO: 5, SEQ ID NO: 7, SEQ ID NO: 9 and SEQ ID NO: 10 and fragments thereof. In particular embodiments, the invention provides the complete sequence of novel reductive dehalogenase proteins (DcpA and DcpB) having the amino acid sequence of SEQ ID NO: 2, SEQ ID NO: 4, SEQ ID NO: 6 and SEQ ID NO: 8 and fragments thereof. The novel reductive dehalogenase genes encodes reductive dehalogenases that are capable of dechlorinating 1,2-dichloropropane to propene.

The present invention further provides for a method of identifying dechlorinating bacterial populations capable of facilitating the reductive dechlorination of organic compounds and in particular the identification of bacterial populations that can dechlorinate 1,2-dichloropropane to propene. Such methods include, but are not limited to, the identification of dechlorinating bacterial populations via the identification of reductive dehalogenase genes, using such methods as hybridization, PCR and Quantitative Real-Time PCR (qPCR). Moreover, such methods may be used to assess and monitor dechlorinating bacterial populations, genes encoding 1,2-dichloropropane reductive dehalogenases and transcripts (such as mRNA transcripts) of the 1,2-dichloropropane reductive dehalogenases at sites contaminated with halogenated compounds and which are amenable to bioremediation practices using dechlorinating bacteria.

### Definitions and Abbreviations

The term reductive dehalogenase is abbreviated “RDase.”

The term “Quantitative Real-Time PCR” is used interchangeably herein with the term “Quantitative PCR” (abbreviated “qPCR”) and is used to mean a method for simultaneous amplification, detection, and quantification of a target polynucleotide using double dye-labeled fluorogenic oligodeoxyribonucleotide probes during PCR and include TaqMan and SYBR Green assays.

As used herein, the term “propene” refers to H₂C═CH—CH₃.

As used herein, the term “1,2-dichloropropane” refers to CH₃—ClCH—CH₂Cl.

“Reductive dehalogenase” or “Reductive dehalogenase enzyme” refers to an enzyme system that is capable of dehalogenating a halogenated straight chain or ring containing organic compound that contains at least one halogen atom. Examples of halogenated organic compounds that may be dehalogenated by a reductive dehalogenase include, but are not limited to, 1,2-dichloropropane, perchloroethylene (Cl₂C═CCl₂), trichloroethylene (Cl₂C═CH—Cl), dichloroethylene (Cl—HC═CH—Cl) and vinyl chloride (H₂C═CH—Cl).

“Dechlorinating bacteria” refers to a bacterial species or organism population that has the ability to remove at least one chlorine atom from a chlorinated organic compound. Examples of dechlorinating bacteria include, but are not limited to members of Dehalococcoides mccartyi, Dehalogenimonas lycanthroporepellens, Dehalobacter restrictus, Sulfurospirillum multivorans, Desulfitobacterium dehalogenans, Geobacter lovleyi, Desulfuromonas chloroethenica, and Desulfuromonas michiganensis.

As referred to herein, “sequence similarity” means the extent to which nucleotide or protein sequences are related. The extent of similarity between two sequences can be based on percent sequence identity and/or conservation. With regard to proteins, “sequence identity” is a comparison of exact amino acid matches, whereas sequence similarity refers to amino acids at a position that have the same physical-chemical properties (i.e. charge, hydrophobicity). Amino acids other than those indicated as conserved may differ in a protein or enzyme so that the percent protein or amino acid sequence similarity between any two proteins of similar function may vary. With regard to polynucleotides, “sequence identity” is a comparison of exact nucleotide matches. Preferably, the sequence identity is at least 80%, preferably at least 85%, more preferably at least 90%, even more preferably at least 95%, even more preferably at least 97%, and most preferably at least 99%, as determined by an alignment scheme.

“Sequence alignment” means the process of lining up two or more sequences to achieve maximal levels of sequence identity (and, in the case of amino acid sequences, conservation), e.g., for the purpose of assessing the degree of sequence similarity or the degree of sequence identity. Methods for aligning sequences and assessing similarity and/or identity are well known in the art. Such methods include for example, the MEGALIGN software Clustal Method, wherein similarity is based on the MEGALIGN Clustal algorithm, ClustalW and ClustalX (Thompson, J., et al. (1997) Nucleic Acid Res. 25:4876-4882) as well as BLASTN, BLASTP, and FASTA (Pearson, et al. (1988) Proc. Natl. Acad. Sci. USA. 85:2444-2448). When using these programs, the preferred settings are those that result in the highest sequence similarity or identity.

### Molecular Biology

In accordance with the present invention there may be employed conventional molecular biology, microbiology, and recombinant DNA techniques within the skill of the art. The general genetic engineering tools and techniques discussed herein, including transformation and expression, the use of host cells, vectors, expression systems, etc., are well known in the art. See, e.g., Sambrook, Fritsch & Maniatis, Molecular Cloning: A Laboratory Manual, Third Edition (2001) Cold Spring Harbor Laboratory Press, Cold Spring Harbor, N.Y. (herein “Sambrook et al. 2001”); DNA Cloning: A Practical Approach, Volumes I and II, Second Edition (D. N. Glover ed. 1995); B. Perbal, A Practical Guide To Molecular Cloning (1984); F. M. Ausubel et al. (eds.), Current Protocols in Molecular Biology, John Wiley & Sons, Inc. (1994).

The term “host cell” means any cell of any organism that is selected, modified, transformed, grown, used, or manipulated in any way, for the production of a substance by the cell, for example the expression by the cell of a gene in this cell, a DNA or RNA sequence, a protein or an enzyme.

A “polynucleotide” or “nucleotide sequence” is a series of nucleotide bases (also called “nucleotides”) in DNA and RNA, and means any chain of two or more nucleotides. A nucleotide sequence typically carries genetic information, including the information used by cellular machinery to make proteins and enzymes. These terms include double or single stranded genomic and cDNA, RNA, any synthetic and genetically manipulated polynucleotides, and both sense and anti-sense polynucleotides (although only sense stands are being represented herein). This includes single- and double-stranded molecules, i.e., DNA-DNA, DNA-RNA and RNA-RNA hybrids, as well as “protein nucleic acids” (PNAs) formed by conjugating bases to an amino acid backbone. This also includes nucleic acids containing modified bases, for example thio-uracil, thio-guanine and fiuoro-uracil.

Polynucleotides may be flanked by natural regulatory sequences, or may be associated with heterologous sequences, including promoters, enhancers, response elements, signal sequences, polyadenylation sequences, introns, 5′- and 3′-non-coding regions, and the like, and may be modified by many means known in the art.

The term “gene” means a DNA sequence that codes for or corresponds to a particular sequence of amino acids which comprise all or part of one or more proteins or enzymes, and may or may not include regulatory DNA sequences, such as promoter sequences, which determine, for example, the conditions under which the gene is expressed.

A “coding sequence” or a sequence “encoding” a polypeptide, protein, enzyme or portion thereof is a nucleotide sequence that, when expressed, results in the production of that polypeptide, protein, enzyme or portion thereof, i.e., the nucleotide sequence encodes an amino acid sequence for that polypeptide, protein, enzyme, or portion thereof. Preferably, the coding sequence is a double-stranded DNA sequence that is transcribed and translated into a polypeptide in a cell in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. If the coding sequence is intended for expression in a eukaryotic cell, a polyadenylation signal and transcription termination sequence will usually be located 3′ to the coding sequence.

Transcriptional and translational control sequences are DNA regulatory sequences, such as promoters, enhancers, terminators, and the like, that provide for the expression of a coding sequence in a host cell. In eukaryotic cells, polyadenylation signals are control sequences.

A “promoter sequence” is a DNA regulatory region capable of binding RNA polymerase in a cell and initiating transcription of a downstream (3′ direction) coding sequence. For purposes of defining this invention, the promoter sequence is bounded at its 3′ terminus by the transcription initiation site and extends upstream (51 direction) to include the minimum number of bases or elements necessary to initiate transcription at levels detectable above background. As described above, promoter DNA is a DNA sequence which initiates, regulates, or otherwise mediates or controls the expression of the coding DNA. A promoter may be “inducible”, meaning that it is influenced by the presence or amount of another compound (an “inducer”). For example, an inducible promoter includes those that initiate or increase the expression of a downstream coding sequence in the presence of a particular inducer compound. A “leaky” inducible promoter is a promoter that provides a high expression level in the presence of an inducer compound and a comparatively very low expression level, and at minimum a detectable expression level, in the absence of the inducer.

The terms “express” and “expression” mean allowing or causing the information in a gene or DNA fragment to become manifest, for example producing a protein by activating the cellular functions involved in transcription and translation of a corresponding gene or DNA sequence. A DNA sequence is expressed in or by a cell to form an “expression product” such as messenger RNA (mRNA) or a protein. The expression product itself, e.g., the resulting protein or enzyme, may also be “expressed” by the cell. A polynucleotide or polypeptide is expressed recombinantly, for example, when it is expressed or produced in a foreign host cell under the control of a foreign or native promoter, or in a native host cell under the control of a foreign promoter.

The term “transformation” means the introduction of a “foreign” (i.e. extrinsic or extracellular) gene, DNA or RNA sequence to a host cell, so that the host cell will express the introduced gene or DNA fragment to produce a desired substance, typically a protein or enzyme coded by the introduced gene or sequence. The introduced gene or sequence, which may also be called a “cloned” or “foreign” gene or sequence, may include regulatory or control sequences, such as start, stop, promoter, signal, secretion, or other sequences used by a cell's genetic machinery. The gene or sequence may include nonfunctional sequences or sequences with no known function. A host cell that receives and expresses introduced DNA or RNA has been “transformed” and is a “transformant” or a “clone.” The DNA or RNA introduced to a host cell can come from any source, including cells of the same genus or species as the host cell, or cells of a different genus or species.

The terms “vector”, “cloning vector” and “expression vector” mean the vehicle by which a DNA or RNA sequence (e.g., a foreign gene) can be introduced into a host cell, so as to transform the host and promote expression (e.g., transcription and translation) of the introduced sequence.

A common type of vector is a “plasmid”, which generally is a self-replicating molecule of double-stranded DNA. A plasmid can readily accept additional (foreign) DNA and can readily be introduced into a suitable host cell. A plasmid vector often contains coding DNA and promoter DNA and has one or more restriction sites suitable for inserting foreign DNA. Promoter DNA and coding DNA may be from the same gene or from different genes, and may be from the same or different organisms. A large number of vectors, including plasmid vectors, have been described for replication and/or expression in a variety of eukaryotic and prokaryotic hosts. Non-limiting examples include pKK plasmids (Clontech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.), and many appropriate host cells, using methods disclosed or cited herein or otherwise known to those skilled in the relevant art. Recombinant cloning vectors will often include one or more replication systems for cloning or expression, one or more markers for selection in the host, e.g. antibiotic resistance, and one or more expression vectors. Routine experimentation in biotechnology can be used to determine which vectors are best suited for use with the present invention. In general, the choice of vector depends on the size of the polynucleotide sequence and the host cells to be used.

The term “expression system” means a host cell and compatible vector under suitable conditions, e.g. for the expression of a protein coded for by foreign DNA carried by the vector and introduced to the host cell. Common expression systems include bacteria (e.g., E. coli and B. subtilis) or yeast (e.g., S. cerevisiae) host cells and plasmid vectors, and insect host cells and Baculovirus vectors. As used herein, a “facile expression system” means any expression system that is foreign or heterologous to a selected polynucleotide or polypeptide, and which employs host cells that can be grown or maintained more advantageously than cells that are native or heterologous to the selected polynucleotide or polypeptide, or which can produce the polypeptide more efficiently or in higher yield. For example, the use of robust prokaryotic cells to express a protein of eukaryotic origin would be a facile expression system. Preferred facile expression systems include E. coli, B. subtilis, and S. cerevisiae, and reductively dechlorinating populations that are easy to cultivate (e.g., Anaeromyxobacter dehalogenans strains and Desulfitobacterium species) as host cells and for any suitable vector.

“Sequence-conservative variants” of a polynucleotide sequence are those in which a change of one or more nucleotides in a given codon position results in no alteration in the amino acid encoded at that position.

As used herein, the terms “isolated” and “purified” are used interchangeably to refer to, for example, polynucleotides, proteins, enzymes or portions thereof that have been removed from their original environment (for example, from their natural environment if they are naturally occurring, or from the host cell if they are produced by recombinant DNA methods). Methods for polypeptide purification are well known in the art and include, but are not limited to, preparative disc-gel electrophoresis, isoelectric focusing, HPLC, reversed-phase HPLC, gel filtration, ion exchange, hydrophobic interaction, affinity, and partition chromatography, and countercurrent distribution. For some purposes, it is preferable to produce the polypeptide in a recombinant system in which the protein contains an additional sequence tag that facilitates purification, such as, but not limited to, a polyhistidine sequence. The polypeptide can then be purified from a crude lysate of the host cell by chromatography on an appropriate solid-phase matrix. Alternatively, antibodies produced against the protein or against peptides derived therefrom can be used as purification reagents. Other purification methods are possible. A purified or isolated polynucleotide or polypeptide may contain less than about 50%, preferably less than about 75%, and most preferably less than about 90%, of the cellular components with which it was originally associated. A “substantially pure” enzyme indicates the highest degree of purity that can be achieved using conventional purification techniques known in the art.

Polynucleotides are “hybridizable” to each other when at least one strand of one polynucleotide can anneal to another polynucleotide under defined stringency conditions. Stringency of hybridization is determined, e.g., by the temperature at which hybridization and/or washing is performed, and b) the ionic strength and polarity (e.g., formamide) of the hybridization and washing solutions, as well as other parameters. Hybridization requires that the two polynucleotides contain substantially complementary sequences; depending on the stringency of hybridization, however, mismatches may be tolerated. Typically, hybridization of two sequences at high stringency (such as, for example, in an aqueous solution of 0.5×SSC at 65° C.) requires that the sequences exhibit some high degree of complementarity over their entire sequence. Conditions of intermediate stringency (such as, for example, an aqueous solution of 2×SSC at 65° C.) and low stringency (such as, for example, an aqueous solution of 2×SSC at 55° C.), require correspondingly less overall complementarity between the hybridizing sequences. (1×SSC is 0.15 M NaCl, 0.015 M Na citrate.) Polynucleotides that “hybridize” to the polynucleotides herein may be of any length. In one embodiment, such polynucleotides are at least 10, preferably at least 15 and most preferably at least 20 nucleotides long. In another embodiment, polynucleotides that hybridize are of about the same length. In another embodiment, polynucleotides that hybridize include those which anneal under suitable stringency conditions and which encode polypeptides, proteins or enzymes having the same function, such as the ability to catalyze a reaction.

### Identification of RDase Genes

In certain embodiments, the present invention provides polynucleotide fragments which may be useful as primers and probes for the identification of genes encoding reductive dehalogenases (RDases). In one embodiment, the invention provides polynucleotide fragments useful for the isolation of RDase genes by aligning conserved regions of full-length protein and DNA sequences of TceA and RDases. Examples of such primers are shown in Table 1 below.

TABLE 1
Polynucleotide fragments

| Primer | Nucleotide Sequence | Target |
|---|---|---|
| RRF2 | 5′-SHMGBMGWGATTTYATGAARR-3′ | RRXFXK motif |
| | SEQ ID NO: 11 | |
| B1R | 5′-CHADHAGCCAYTCRTACCA-3′ | WYEW motif |
| | SEQ ID NO: 12 | |

*Abbreviations of degenerate nucleotides: R = A/G; K = G/T; M = A/C; S = C/G; W = A/T; Y = C/T; B = C/G/T; D = A/G/T; V = A/C/G; H = A/C/T.

The invention also provides PCR primer pairs and probes useful in the identification of RDase genes, as well as a number of polynucleotide fragments encoding at least a portion of several RDases. The PCR primer pairs, probes and polynucleotide fragments of the present invention may be used to isolate cDNAs and genes encoding homologous proteins from the same or other dechlorinating bacteria species.

Isolation of homologous genes using sequence-dependent protocols is well-known in the art. Examples of sequence-dependent protocols include, but are not limited to, methods of nucleic acid hybridization, and methods of DNA and RNA amplification as exemplified by various uses of nucleic acid amplification technologies (e.g., PCR, ligase chain reaction).

For example, genes encoding other RDases, either as cDNAs or genomic DNAs, could be isolated directly by using all or a portion of the instant polynucleotide fragments as DNA hybridization probes to screen libraries from any desired dechlorinating bacterial population employing methodology well known to those skilled in the art. Specific oligonucleotide probes based upon the instant nucleic acid sequences can be designed and synthesized by methods known in the art (see, e.g., Sambrook, et al. 2001). Moreover, an entire sequence can be used directly to synthesize DNA probes by methods known to the skilled artisan such as random primer DNA labeling, nick translation, end-labeling techniques, or RNA probes using available in vitro transcription systems. In addition, specific primers can be designed and used to amplify a part or all of the instant sequences. The resulting amplification products can be labeled directly during amplification reactions or labeled after amplification reactions, and used as probes to isolate full-length cDNA or genomic fragments under conditions of appropriate stringency.

In addition, two short segments of the instant polynucleotide fragments may be used in PCR protocols to amplify longer nucleic acid fragments encoding homologous genes from DNA or RNA. The PCR may also be performed on a library of cloned nucleic acid fragments to identify nucleotide sequences encoding bacterial reductive dehalogenases including, for example, dcpA.

Alternatively, the second primer sequence may be based upon sequences derived from the cloning vector. For example, the skilled artisan can follow the RACE protocol (Frohman, et al. (1988) Proc. Natl. Acad. Sci. USA 85:8998-9002) to generate cDNAs by using PCR to amplify copies of the region between a single point in the transcript and the 3′ or 5′ end. Primers oriented in the 3′ and 5′ directions can be designed from the instant sequences. Using commercially available 3′ RACE or 5′ RACE systems, specific 3′ or 5′ cDNA fragments can be isolated (Ohara, et al. (1989) Proc. Natl. Acad. Sci. USA 86:5673-5677; Loh, et al. (1989) Science 243:217-220). Products generated by the 3′ and 5′ RACE procedures can be combined to generate full-length cDNAs (Frohman and Martin (1989) Techniques 1:165). Consequently, a polynucleotide comprising a nucleotide sequence of about 30 or more contiguous nucleotides derived from a nucleotide sequence, such as for example, the nucleotide sequences selected from the group consisting of SEQ ID NO: 1, SEQ ID NO: 3, SEQ ID NO: 5, SEQ ID NO: 7, SEQ ID NO: 9 and SEQ ID NO: 10 and the complement of such nucleotide sequences may be used in such methods to obtain a nucleic acid fragment encoding a substantial portion of an amino acid sequence of a polypeptide.

### Identification, Use and Expression of RDase Polypeptides

In certain additional embodiments, the present invention provides a method of obtaining a polynucleotide fragment encoding a RDase polypeptide, preferably a substantial portion of a RDase polypeptide, comprising the steps of: (i) synthesizing a pair of oligonucleotide primers, wherein each oligonucleotide primer comprises preferably at least about 10, more preferably at least about 15, and still more preferably at least about 25 contiguous nucleotides derived from a nucleotide sequence selected from the group consisting of SEQ ID NO: 1, SEQ ID NO: 3, SEQ ID NO: 5, SEQ ID NO: 7, SEQ ID NO: 9 and SEQ ID NO: 10 or the complement of these sequences; and (ii) amplifying a polynucleotide fragment (preferably a cDNA inserted in a cloning vector) using the oligonucleotide primer pair. The amplified polynucleotide fragment preferably will encode a portion of a RDase polypeptide that occurs between the two primers. In certain embodiments, the oligonucleotide primers have at least 85% and more preferably at least 90%, more preferably at least 95%, more preferably at least 97% and still more preferably at least 99% sequence identity across the length of the primer to a polynucleotide sequence selected from the group consisting of SEQ ID NO: 1, SEQ ID NO: 3, SEQ ID NO: 5, SEQ ID NO: 7, SEQ ID NO: 9, SEQ ID NO: 10, SEQ ID NO: 15, SEQ ID NO: 16, SEQ ID NO: 17, SEQ ID NO: 18, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 55, SEQ ID NO: 56, and SEQ ID NO: 57.

In one embodiment, the availability of the instant nucleotide and deduced amino acid sequences facilitates immunological screening of cDNA expression libraries. Synthetic peptides representing portions of the instant amino acid sequences may be synthesized. These peptides can be used to immunize animals to produce polyclonal or monoclonal antibodies with specificity for peptides or proteins comprising the amino acid sequences. These antibodies can then be used to screen cDNA expression libraries to isolate full-length cDNA clones of interest (see e.g., Sambrook, et al. 2001).

In another embodiment, this invention concerns viruses and host cells comprising either the recombinant expression vectors as described herein or any one of the isolated polynucleotides of the present invention described herein. Examples of host cells which can be used to practice the present invention include, but are not limited to, yeast, bacteria and insect.

Plasmid vectors comprising the instant isolated polynucleotide may be constructed. The choice of plasmid vector is dependent upon the method that will be used to transform a host organism, e.g., yeast, bacterial cell or insect. The skilled artisan is well aware of the genetic elements that must be present on the plasmid vector in order to successfully transform, select and propagate host cells containing the recombinant expression vector. The skilled artisan will also recognize that different independent transformation events will result in different levels and patterns of expression (Jones, et al. (1985) EMBO J. 4:2411-2418; De Almeida, et al. (1989) Mol. Gen. Genetics 218:78-86), and thus that multiple events must be screened in order to obtain lines displaying the desired expression level and pattern. Such screening may be accomplished by Southern analysis of DNA, Northern analysis of mRNA expression, Western analysis of protein expression, or phenotypic analysis.

### Genetic Mapping

The isolated polynucleotides of the present invention may be used as probes for the genetic and physical mapping of the genes they are a part of, and may further be used as markers for traits linked to those genes. Such information may be useful in the art to identify and develop strains of dechlorinating bacteria capable of reducing 1,2-dichloropropane and other chloroorganic contaminants. For example, the instant polynucleotide fragments may be used as probes to detect restriction fragment length polymorphisms (RFLPs) that identify bacterial populations with the dechlorinating activity of interest. Southern blots (see, e.g., Sambrook, et al. 2001) of restriction-digested bacterial genomic DNA may be probed with the polynucleotide fragments of the instant invention. The resulting banding patterns may then be subjected to genetic analyses using computer programs such as MapMaker (Lander, et al. (1987) Genomics 1:174-181) to construct a genetic map.

The isolated polynucleotide fragments may also be used to probe Southern blots containing restriction endonuclease-treated genomic DNAs of a set of individuals representing parent and progeny of a defined genetic cross. Segregation of the DNA polymorphisms is noted and used to calculate the position of the instant polynucleotide sequence in the genetic map previously obtained using this population (Botstein, et al. (1980) Am. J. Hum. Genet. 32:314-331).

Additionally, the isolated polynucleotides of the present invention may be used in a variety of polynucleotide amplification-based methods of genetic and physical mapping. Examples include allele-specific amplification (Kazazian (1989) J. Lab. Clin. Med. 11:95-96), polymorphism of PCR-amplified fragments (CAPS; Sheffield, et al. (1993) Genomics 16:325-332), allele-specific ligation (Landegren, et al. (1988) Science 241:1077-1080), nucleotide extension reactions (Sokolov (1990) Polynucleotide Res. 18:3671), Radiation Hybrid Mapping (Walter, et al. (1997) Nat. Genet. 7:22-28) and Happy Mapping (Dear and Cook (1989) Polynucleotide Res. 17:6795-6807). For these methods, the sequence of a polynucleotide fragment is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant polynucleotide sequence. This, however, is generally not necessary for mapping methods.

### Hybridization Techniques for the Detection of Dechlorinating Bacteria

In another embodiment, the invention provides a method of detecting dechlorinating bacteria using the polynucleotides disclosed herein as hybridization probes. The probe length can vary from 5 bases to thousands of bases. Preferably however, the probe is at least 10, more preferably at least 15 and most preferably at least 20 nucleotides in length. Probes may also be, for example, about 100, 200, 300, 400, or 500 nucleotides in length. Only part of the probe molecule need be complementary to the nucleic acid sequence to be detected and the complementary portion need not be identical. For example, the probe may have at least 85% and more preferably at least 90%, even more preferably at least 95%, even more preferably at least 97% and still more preferably at least 99% sequence identity across the length of the probe to the nucleic acid sequence to be detected. Hence, all or part of the aforementioned lengths may be complementary to the polynucleotide sequence to be detected. The probe may be RNA or DNA or a synthetic nucleic acid. In each instance a probe will contain a sequence sufficiently complementary to the nucleic acid from the dechlorinating bacteria to be detected, and that will permit hybridization between the probe and the subject DNA.

In certain embodiments the probe is a polynucleotide that is substantially complementary to a fragment or the entire polynucleotide sequence of a gene encoding a RDase including, for example, dcpA. In a preferred embodiment, the probe may be selected from a fragment or an entire polynucleotide selected from the group consisting of SEQ ID NO: 1, SEQ ID NO: 3, SEQ ID NO: 5, SEQ ID NO: 7, SEQ ID NO: 9 and SEQ ID NO: 10.

Hybridization methods are well known in the art (see, e.g., Sambrook, et al. 2001). Typically, the probe and sample are mixed under conditions that permit nucleic acid hybridization. This involves contacting the probe and sample in the presence of an inorganic or organic salt under the proper concentration and temperature conditions. The probe and sample nucleic acids must be in contact for a sufficient time that any possible hybridization between the probe and sample nucleic acid may occur. The concentration of probe or target in the mixture will determine the time necessary for hybridization to occur. The higher the probe or target concentration, the shorter the hybridization incubation time needed.

In certain embodiments, hybridization assays may be conducted directly on bacterial lysates, without the need to extract the nucleic acids. This eliminates several steps from the sample-handling process and speeds up the assay. To perform such assays on crude cell lysates, a chaotropic agent is typically added to the cell lysates prepared as described above. The chaotropic agent stabilizes nucleic acids by inhibiting nuclease activity. Furthermore, the chaotropic agent allows sensitive and stringent hybridization of short oligonucleotide probes to RNA at room temperature (Van Ness and Chen (1991) Nucl. Acids Res. 19:5143-5151). Suitable chaotropic agents include guanidinium chloride, guanidinium thiocyanate, sodium thiocyanate, lithium tetrachloroacetate, sodium perchlorate, rubidium tetrachloroacetate, potassium iodide, and cesium trifluoroacetate, among others. Typically, the chaotropic agent will be present at a final concentration of about 3 M. If desired, one can add formamide to the hybridization mixture, typically 30-50% (v/v).

Various hybridization solutions can be employed. Typically, these comprise from about 20 to 60% volume, preferably 30%, of a polar organic solvent. A common hybridization solution comprises about 30-50% v/v formamide, about 0.15 to 1 M sodium chloride, about 0.05 to 0.1 M buffer, such as sodium citrate, Tris-HCl, PIPES or HEPES (pH range about 6-9), about 0.05 to 0.2% detergent, such as sodium dodecylsulfate, and between 0.5-20 mM EDTA, FICOLL™ (Amersham Biosciences, Piscataway, N.J.) (about 300-500 kDa), polyvinylpyrrolidone (about 250-500 kDa), and serum albumin. Also included in the typical hybridization solution, will be from about 0.1 to 5 mg/ml, unlabeled carrier nucleic acids, e.g., fragmented calf thymus or salmon sperm DNA, or yeast RNA, and optionally from about 0.5 to 2% wt/vol glycine. Other additives may also be included, such as volume exclusion agents, which include a variety of polar water-soluble or swellable agents, such as polyethylene glycol, anionic polymers such as polyacrylate or polymethylacrylate, and anionic saccharidic polymers, such as dextran sulfate.

Hybridization is adaptable to a variety of assay formats. One of the most suitable is the sandwich assay format. The sandwich assay is particularly adaptable to hybridization under non-denaturing conditions. A primary component of a sandwich-type assay is a solid support. The solid support has adsorbed to it or covalently coupled to it immobilized nucleic acid probe that is unlabeled and complementary to one portion of the nucleic acid to be detected, e.g., nucleic acid encoding for a reductive dehalogenase including, for example, dcpA. Probes particularly useful in the present embodiment are those polynucleotides which are substantially complementary to a fragment or the entire polynucleotide sequence of a gene encoding a RDase such as DcpA, and in particular those which are substantially complementary to SEQ ID NO: 1 or SEQ ID NO: 3.

The sandwich assay may be encompassed in an assay kit. A kit may include a first component for the collection of samples from soil or groundwater, such as vials for containment, and buffers for the disbursement and lysis of the sample. A second component may include media in either dry or liquid form for the hybridization of target and probe polynucleotides, as well as for the removal of undesirable and nonduplexed forms by washing. A third component includes a solid support (dipstick) upon which is fixed or to which is conjugated unlabeled nucleic acid probe(s) that is (are) complementary to a part of a nucleic acid encoding for a reductive dehalogenase of the species of bacteria being tested such as, for example, dcpA.

### PCR Based Detection of Dechlorinating Bacteria

In an another embodiment, the polynucleotides of the present invention may be used as primers in primer directed nucleic acid amplification, i.e., PCR, to detect the presence of the target gene(s) in the dechlorinating wild type bacteria. Methods of PCR primer design are well known in the art (see, e.g., Sambrook, et al. 2001; Herndon, Va. and Rychlik, W. (1993 In White, B. A. (ed.), Methods in Molecular Biology, Vol. 15, pp 31-39, PCR Protocols: Current Methods and Applications. Humania Press, Inc., Totowa, N.J.; see also, U.S. Pat. Nos. 4,683,195; 4,683,2020; 4,965,188; and 4,800,159, which are hereby incorporated by reference).

Typically, detection of dechlorinating bacteria, such as Dehalococcoides strains including Dehalococcoides (Dhc) mccartyi strains RC and KS, using PCR involves the amplification of DNA or cDNA obtained from a sample suspected of having dechlorinating activity. The isolated DNA or cDNA (from mRNA) is amplified using a pair of oligonucleotide primers, wherein one primer (a forward primer) binds to the coding strand of the template and the other primer (a reverse primer) binds to the complementary strand of the template, thus creating two copies of the target region in each PCR cycle. A primer refers to an oligonucleotide that can be extended with a DNA polymerase using monodeoxyribonucleoside triphosphates and a nucleic acid that is used as a template. This primer preferably has a 3′ hydroxyl group on an end that is facing the 5′ end of the template nucleic acid when it is hybridized with the template.

A set of primers refers to a combination or mixture of at least a first (forward) and a second (reverse) primer. The first primer can be extended using the template nucleic acid while forming an extension product in such a way that the second primer can hybridize with this extension product in a region of the extension product that lies in the 3′ direction of the extendable end of the first primer. The extendable end of the second primer points in the 5′ direction of the extension product of the first primer. Examples of primers that are suitable for performing the polymerase chain reaction (PCR) and that meet this definition are described in European Patent Application No. 0201184, which is hereby incorporated by reference. Typical amplicons range in size from 25 by to 2000 by (see, e.g., U.S. Pat. No. 6,518,025). Larger sized amplicons can be obtained, typically using specialized conditions or modified polymerases.

The primers and probes of the present invention are designed to be specific to regions of the dcpA and/or dcpB genes identified herein. Useful primers and probes include, but are not limited to, those having the polynucleotide sequence of any one of SEQ ID NOS: 15-18, SEQ ID NOS: 20-50 and SEQ ID NOS: 55-57. In one preferred embodiment, the first primer is the polynucleotide of SEQ ID NO: 15 and the second primer is the polynucleotide of SEQ ID NO: 16. In another preferred embodiment, the first primer is the polynucleotide of SEQ ID NO: 17 and the second primer is the polynucleotide of SEQ ID NO: 16. In another preferred embodiment, the first primer is the polynucleotide of SEQ ID NO: 18 and the second primer is the polynucleotide of SEQ ID NO: 16. In another preferred embodiment, the first primer is the polynucleotide of SEQ ID NO: 21 and the second primer is the polynucleotide of SEQ ID NO: 22. In another preferred embodiment, the first primer is the polynucleotide of SEQ ID NO: 24 and the second primer is the polynucleotide of SEQ ID NO: 25. In another preferred embodiment, the first primer is the polynucleotide of SEQ ID NO: 27 and the second primer is the polynucleotide of SEQ ID NO: 28. In another preferred embodiment, the first primer is the polynucleotide of SEQ ID NO: 30 and the second primer is the polynucleotide of SEQ ID NO: 31. In another preferred embodiment, the first primer is the polynucleotide of SEQ ID NO: 33 and the second primer is the polynucleotide of SEQ ID NO: 34. In another preferred embodiment, the first primer is the polynucleotide of SEQ ID NO: 36 and the second primer is the polynucleotide of SEQ ID NO: 37. In another preferred embodiment, the first primer is the polynucleotide of SEQ ID NO: 39 and the second primer is the polynucleotide of SEQ ID NO: 40. In another preferred embodiment, the first primer is the polynucleotide of SEQ ID NO: 42 and the second primer is the polynucleotide of SEQ ID NO: 43. In another preferred embodiment, the first primer is the polynucleotide of SEQ ID NO: 45 and the second primer is the polynucleotide of SEQ ID NO: 46. In another preferred embodiment, the first primer is the polynucleotide of SEQ ID NO: 48 and the second primer is the polynucleotide of SEQ ID NO: 49. In another preferred embodiment, the first primer is the polynucleotide of SEQ ID NO: 55 and the second primer is the polynucleotide of SEQ ID NO: 56.

Following amplification, the products of PCR may be detected using any one of a variety of PCR detection methods are known in the art including standard non-denaturing gel electrophoresis (e.g., acrylamide or agarose), denaturing gradient gel electrophoresis, and temperature gradient gel electrophoresis. Standard non-denaturing gel electrophoresis is the simplest and quickest method of PCR detection, but may not be suitable for all applications.

### Quantitative Real-Time PCR Based Enumeration of Dechlorinating Bacteria

In yet another embodiment, the invention provides a method of detecting and enumerating dechlorinating bacteria using Quantitative Real-Time PCR (“qPCR”). Quantitative Real-Time PCR is a further enhancement to the standard PCR, described above. Quantitative Real-Time PCR allows contemporaneous quantification of a sample of interest, for example a bacteria population having a polynucleotide sequence of interest.

In qPCR, a fluorogenically labeled oligonucleotide probe is used in addition to the primer sets which are employed in standard PCR. In qPCR, the probe anneals to a sequence on the target DNA found between a first (forward, 5′ primer) and second (reverse, 3′ primer) PCR primer binding sites and consists of an oligonucleotide with a 5′-reporter dye (e.g., FAM, 6-carboxyfluorescein) and a quencher dye [e.g., TAMRA, 6-carboxytetramethylrhodamine, black hole quencher (BHQ)] which quenches the emission spectra of the reporter dye as long as both dyes are attached to the probe. The probe signals the formation of PCR amplicons by a process involving the polymerase-induced nucleolytic degradation of the double-labeled fluorogenic probe that anneals to the target template at a site between the two primer recognition sequences (see, e.g., U.S. Pat. No. 6,387,652).

The measurement of the released fluorescent emission following each round of PCR amplification (Heid et al., (1996) Genome Research, 6:986-994) thus forms the basis for quantifying the amount of target nucleic acid present in a sample at the initiation of the PCR reaction. Since the exponential accumulation of the fluorescent signal directly reflects the exponential accumulation of the PCR amplification product, this reaction is monitored in real time. Hardware, such as the model 7500 fast, 7700, model 7900HT, and Viia7 Sequence Detection Systems, available from Applied Biosystems (Foster City, Calif.) can be used to automate the detection and quantitative measurement of these signals, which are stoichiometrically related to the quantities of amplicons produced. From the output data of the qPCR, quantification from a reliable back calculation to the input target DNA sequence is possible using standard curves generated with known amounts of template DNA.

Primers and probes useful in qPCR identification and quantification of a bacteria population having a polynucleotide sequence of interest may be designed to correspond to the polynucleotide of interest. In one embodiment of the present invention, primers and probes useful in qPCR correspond to regions of the dcpA and dcpB genes identified herein. Primers and probes useful in the present embodiment include, but are not limited to, those having the polynucleotide sequence of any one of SEQ ID NOS: 15-18, SEQ ID NOS: 20-50, and SEQ ID NOS: 55-57. Useful qPCR probes include, but are not limited to, those polynucleotides which hybridize to any one of SEQ ID NO: 1, SEQ ID NO: 3, SEQ ID NO: 5, SEQ ID NO: 7, SEQ ID NO: 9 and SEQ ID NO: 10 or the complement of any of these sequences.

In some embodiments, a first oligonucleotide primer for qPCR comprises a sequence selected from the group consisting of SEQ ID NO: 15, SEQ ID NO: 17, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, and SEQ ID NO: 55; a second oligonucleotide primer comprises a sequence selected from the group consisting of SEQ ID NO: 16, SEQ ID NO: 22, SEQ ID NO: 25, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, and SEQ ID NO: 56; and a probe comprises a sequence selected from the group consisting of SEQ ID NO: 20, SEQ ID NO: 23, SEQ ID NO: 26, SEQ ID NO: 29, SEQ ID NO: 32, SEQ ID NO: 35, SEQ ID NO: 38, SEQ ID NO: 41, SEQ ID NO: 44, SEQ ID NO: 47, SEQ ID NO: 50, and SEQ ID NO: 57.

In one preferred embodiment, the PCR primer pair and probe for use in qPCR consist of a first (forward) primer having the polynucleotide sequence of SEQ ID NO: 18, a second (reverse) primer having the polynucleotide sequence of SEQ ID NO: 16 and probe having the polynucleotide sequence of SEQ ID NO: 20. In another preferred embodiment, the PCR primer pair and probe for use in qPCR consist of a first (forward) primer having the polynucleotide sequence of SEQ ID NO: 21, a second (reverse) primer having the polynucleotide sequence of SEQ ID NO: 22 and probe having the polynucleotide sequence of SEQ ID NO: 23. In another preferred embodiment, the PCR primer pair and probe for use in qPCR consist of a first (forward) primer having the polynucleotide sequence of SEQ ID NO: 24, a second (reverse) primer having the polynucleotide sequence of SEQ ID NO: 25 and probe having the polynucleotide sequence of SEQ ID NO: 26. In another preferred embodiment, the PCR primer pair and probe for use in qPCR consist of a first (forward) primer having the polynucleotide sequence of SEQ ID NO: 27, a second (reverse) primer having the polynucleotide sequence of SEQ ID NO: 28 and probe having the polynucleotide sequence of SEQ ID NO: 29. In another preferred embodiment, the PCR primer pair and probe for use in qPCR consist of a first (forward) primer having the polynucleotide sequence of SEQ ID NO: 30, a second (reverse) primer having the polynucleotide sequence of SEQ ID NO: 31 and probe having the polynucleotide sequence of SEQ ID NO: 32. In another preferred embodiment, the PCR primer pair and probe for use in qPCR consist of a first (forward) primer having the polynucleotide sequence of SEQ ID NO: 33, a second (reverse) primer having the polynucleotide sequence of SEQ ID NO: 34 and probe having the polynucleotide sequence of SEQ ID NO: 35. In another preferred embodiment, the PCR primer pair and probe for use in qPCR consist of a first (forward) primer having the polynucleotide sequence of SEQ ID NO: 36, a second (reverse) primer having the polynucleotide sequence of SEQ ID NO: 37 and probe having the polynucleotide sequence of SEQ ID NO: 38. In another preferred embodiment, the PCR primer pair and probe for use in qPCR consist of a first (forward) primer having the polynucleotide sequence of SEQ ID NO: 39, a second (reverse) primer having the polynucleotide sequence of SEQ ID NO: 40 and probe having the polynucleotide sequence of SEQ ID NO: 41. In another preferred embodiment, the PCR primer pair and probe for use in qPCR consist of a first (forward) primer having the polynucleotide sequence of SEQ ID NO: 42, a second (reverse) primer having the polynucleotide sequence of SEQ ID NO: 43 and probe having the polynucleotide sequence of SEQ ID NO: 44. In another preferred embodiment, the PCR primer pair and probe for use in qPCR consist of a first (forward) primer having the polynucleotide sequence of SEQ ID NO: 45, a second (reverse) primer having the polynucleotide sequence of SEQ ID NO: 46 and probe having the polynucleotide sequence of SEQ ID NO: 47. In another preferred embodiment, the PCR primer pair and probe for use in qPCR consist of a first (forward) primer having the polynucleotide sequence of SEQ ID NO: 48, a second (reverse) primer having the polynucleotide sequence of SEQ ID NO: 49 and probe having the polynucleotide sequence of SEQ ID NO: 50. In another preferred embodiment, the PCR primer pair and probe for use in qPCR consist of a first (forward) primer having the polynucleotide sequence of SEQ ID NO: 55, a second (reverse) primer having the polynucleotide sequence of SEQ ID NO: 56 and probe having the polynucleotide sequence of SEQ ID NO: 57.

Quantitative Real-Time PCR may be used to identify and quantify a population of dechlorinating bacteria having a polynucleotide sequence of interest by first isolating DNA from a sample suspected of having dechlorinating activity using any one of the methods known in the art (see e.g., He, J. et al. (2003) Appl. Environ. Microbiol. 65:485-495). The isolated DNA may be amplified using qPCR by contacting the sample with any one of the probes described above, and any one of the primer pairs described above. Preferably, the probe is fluorogenically labeled. For example, the probe is labeled with 6-carboxy-fluorescein (FAM) as a reporter fluorochrome on the 5′ end, and N,N,N′,N′-tetramethyl-6-carboxy-rhodamine (TAMRA) as quencher on the 3′ end. The isolated DNA sample is subjected to qPCR using any one of the qPCR protocols known in the art, such as the qPCR protocol described in U.S. Provisional Application No. 60/474,831, which is hereby incorporated by reference. During the course of PCR the fluorescent signal generated by the reaction may be continuously monitored using detection hardware, such as the model 7500 fast, 7700 and model 7900HT Sequence Detection Systems, available from Applied Biosystems (Foster City, Calif.).

The amount of dechlorinating bacteria containing the polynucleotide sequence of interest, present in the sample may be determined using qPCR, by comparing the results of the qPCR assay described above to a calibration curve. A calibration curve (log DNA concentration versus arbitrarily set cycle threshold value, C_{T}) may be obtained using serial dilutions of DNA of known concentration or gene copy numbers. The C_{T} values obtained for each sample may be compared with the standard curve to determine the abundance of Dehalococcoides gene targets. Using an average molecular weight of 660 for a base pair in dsDNA (or 330 in the case of cDNA), one reductive dehalogenase gene operon per Dehalococcoides genome, and a genome size of 1.5 Mbp (www.tigr.org), the following equation may be used to ascertain the number of Dehalococcoides-derived reductive dehalogenase gene copies that were present in the DNA obtained from 1 mL of the dechlorinating enrichment culture:

$$\frac{\begin{matrix}Reductive dehalogenaise gene \\ copies \text{/} ml = DNA ( μg / ml \times 6.023 \times {10}^{23}\end{matrix} }{( 1.5 \times {10}^{6} \times 660 ) \times {10}^{6} )}$$

### EXAMPLES

The present invention is further exemplified in the following Examples, which are presented by way of illustration, not by way of limitation.

### Example 1

### Isolation of 1,2-Dichloropropane Dechlorinating Cultures

The 1,2-dichloropropane dechlorinating cultures Dehalococcoides (Dhc) mccartyi strain RC and strain KS were derived from the Red Cedar River near Okemos, Mich., and the King Salmon River, Ak., respectively (Löffler, F. E., et al (1997) Appl. Environ. Microbiol. 63:2870-2875; Ritalahti, K. M., and F. E. Löffler (2004) Appl. Environ. Microbiol. 70:4088-4095). The Red Cedar River has no known anthropogenic sources of chlorinated solvents but is located in an agricultural area. The King Salmon River sediment had reported hydrocarbon contamination. Both cultures are non-methanogenic and have been maintained in reduced mineral salts medium (Löffler, F. E., et al (1997) Appl. Environ. Microbiol. 63:2870-2875; Ritalahti, K. M., and F. E. Löffler (2004) Appl. Environ. Microbiol. 70:4088-4095) for more than 10 years. Briefly, the cultures were grown in 160 mL serum bottles containing 100 mL of defined, completely synthetic reduced mineral salts medium (Löffler, F. E et al. (1996). Appl. Environ. Microbiol. 62:3809-3813) amended with 5 mM acetate, 30 mM bicarbonate (pH 7.2), 0.2 mM Na₂S×9H₂O, resazurin (0.25 mg/L), vitamins (Wolin, E. A., et al. (1963) J. Biol. Chem. 238:2882-2886), 5 mL H₂ and 5 μL of neat 1,2-dichloropropane (Supelco, Bellefonte, Pa.). 1,2-dichloropropane and propene concentrations were measured in headspace and aqueous samples using a HP 6890 gas chromatograph (GC) equipped with a HP-624 column (60 m length, 0.32 mm diameter; film thickness of 1.8 μm nominal) and a flame ionization detector (FID) as previously described (Amos B. K., et al. (2007) Environ. Sci. Technol. 41:963-970).

### Example 2

### Preparation of cDNA from 1,2-Dichloropropane Dechlorinating Cultures

Biomass was collected from 10-20 mL of culture suspensions from each of the RC and KS cultures described in Example 1, and the cells were harvested by vacuum filtration onto a Durapore hydrophilic polyvinylidene fluoride membrane (25 mm diameter and 0.22 μm pore size) (Millipore, Billerica, Mass.). RNA extraction, cDNA synthesis and purification were performed as described previously (Ritalahti, K. M., et al. (2010) In K. N. Timmis (ed.), Handbook of Hydrocarbon and Lipid Microbiology. Springer Berlin Heidelberg 32:3671-3685). Briefly, RNA was extracted following the TRIzol Max Bacterial RNA Isolation Kit protocol (Invitrogen, Carlsbad, Calif., USA) provided by the manufacturer. To assess transcript loss, 1×10¹¹ luciferase mRNA molecules were added (Promega, Madison, Wis., USA) before the lysis step as an internal standard to account for RNA loss during extraction, reverse transcription, DNase treatment and cDNA purification. Two rounds of DNase treatment (Epicentre, Madison, Wis., USA) were performed as described previously (Ritalahti, K. M., et al. (2010) In K. N. Timmis (ed.), Handbook of Hydrocarbon and Lipid Microbiology. Springer Berlin Heidelberg 32:3671-3685). To confirm that the RNA samples were not contaminated with genomic DNA, 2 μl, of cDNA were subjected to PCR (20 μL total reaction volume) with general bacterial 16S rRNA gene-targeted primers Bac8F (5′-AGAGTTTGATCCTGGCTCAG-3′; SEQ ID NO: 51) and Bac1541R (5′-AAGGAGGTGATCCAGCCGCA-3′; SEQ ID NO: 52) as described previously (Löffler, F. E., et al. (2000) Appl. Environ. Microbiol. 66:1369-1374).

The RNA was quantified using the Nanodrop (Thermo Fisher Scientific, Wilmington, Del., USA) and RNA integrity was verified with the Bioanalyzer (Agilent, Palo Alto, Calif., USA). The DNA-free RNA was concentrated using RNeasy MinElute spin columns (Qiagen, Valencia, Calif., USA) to a volume of approximately 15 μL, which was used for cDNA synthesis with random hexamers as described previously (Ritalahti, K. M., et al. (2010) In K. N. Timmis (ed.), Handbook of Hydrocarbon and Lipid Microbiology. Springer Berlin Heidelberg 32:3671-3685). The cDNA was cleaned with a PCR purification kit (Qiagen, Valencia, Calif., USA).

### Example 3

### Reductive Dehalogenase Genes from cDNA Isolated from 1,2-Dichloropropane Dechlorinating Cultures

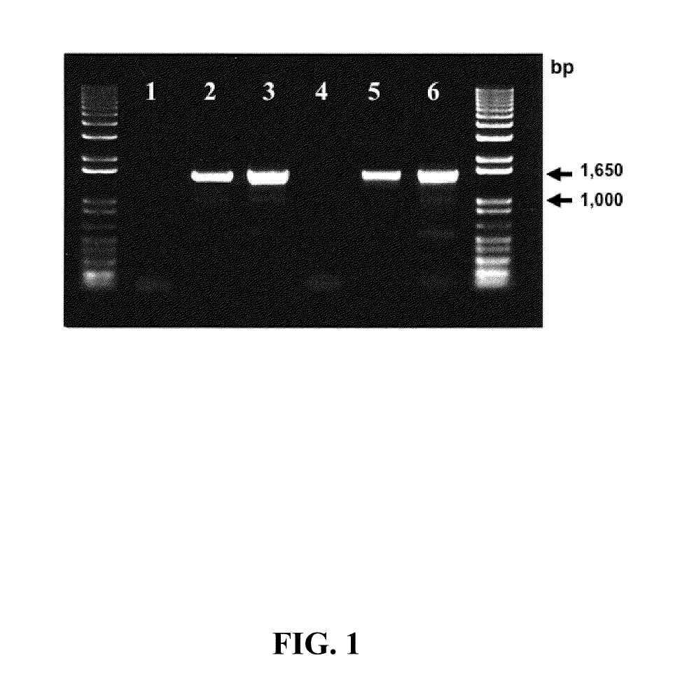

One tenth of the final 20 μL volume of cDNA (prepared as described in Example 2) was used as a template for PCR with primers RRF2 (SEQ ID NO: 11) and B1R (SEQ ID NO: 12), which are degenerate primers for reductive dehalogenase genes (Krajmalnik-Brown, R., et al. (2004) Appl. Environ. Microbiol. 70:6347-6351). The PCR amplicons (approximately 1,500 bp) were resolved by electrophoresis on a 1% (wt/vol) agarose gel prepared with TAE buffer (40 mM Tris base in 20 mM acetic acid, 1 mM EDTA, pH 8.5) and stained with ethidium bromide (1 μg/mL). A picture of the PCR amplicons on the agarose gel is shown in FIG. 1. As explained above, all of the PCR reactions were performed with the B1R and RRF2 degenerate PCR primers. Lanes 1-3 of FIG. 1 correspond to samples from Dhc mccartyi strain RC cultures, and lanes 4-6 correspond to samples from Dhc mccartyi strain KS cultures. Lanes 1 and 4 are “no reverse transcriptase” controls and lanes 3 and 6 correspond to PCR positive controls performed with genomic DNA from the respective cultures. Lanes 2 and 5 are amplicons from PCR reactions using the RRF2 and B1R primers with cDNA prepared from the Dhc mccartyi strain RC and strain KS cultures, respectively, as template DNA. A 1 Kb Plus Ladder from Invitrogen is shown on both ends of the agarose gel. As shown in FIG. 1, no amplification occurred in the RNA sample with no reverse transcriptase control, confirming that DNA had been removed from the RNA pool, and that the resulting cDNA was not contaminated with genomic DNA.

The PCR products were cloned in the pCRII TOPO vector and transformed into E. coli TOP'10 competent cells (TOPO TA cloning kit, Invitrogen). With this methodology, cDNA clone libraries were established and 200 clones were selected and screened with primers targeting the cloning vector that are provided in the Invitrogen TOPO TA cloning kit: M13F (5′-GTAAAACGACGGCCAGT-3′; SEQ ID NO: 13) and M13R (5′-GGAAACAGCTATGACCATG-3′; SEQ ID NO: 14).

Clones with inserts of the correct size (˜1,500 bp) were grown overnight at 37° C. in 3 mL of Lysogeny Broth (LB) medium with 100 μg/mL ampicillin. Glycerol stocks of positive clones were prepared by adding 600 μL of culture suspension to 300 μL of 60% glycerol in 1.5 mL cryovials and stored at −80° C. In addition, 1.5 mL of the E. coli cell suspensions were centrifuged, the supernatants decanted and the pellets stored at −20° C. before plasmid extraction. The QIAprep Spin Miniprep kit (Qiagen, Valencia, Calif., USA) was used for plasmid isolation and the inserts were sequenced using primers M13F (SEQ ID NO: 13) and M13R (SEQ ID NO: 14). The SeqMan II software (DNASTAR, Lasergene version 7) was used for sequence assembly. ClustalW/JALVIEW web-browser plugins were used for sequence alignment and visualization. The assembled contigs for putative RDase sequences were blasted in a nucleotide-nucleotide search against the nr (non-redundant) NCBI database (blastn) (http://www.ncbi.nlm.nih.gov/BLAST/). The top hit was to a portion of the genome of Dehalogenimonas lykanthroporepellens strain BL-DC-9 that encodes a reductive dehalogenase.

### Example 4

### Sequences of the dcpA and dcpB Genes and the DcpA and DcpB Proteins

The positive clones described in Example 3 had a single 1,486 by long insert that included a partial RDase A gene fragment and a partial RDase B gene fragment, indicating that both genes were co-transcribed. The RDase A gene fragment in the RC and KS strains included 1,433 nucleotides, and a partial gene fragment for the RDase B gene of 35 nucleotides began 18 nucleotides downstream of the RDase A stop codon.

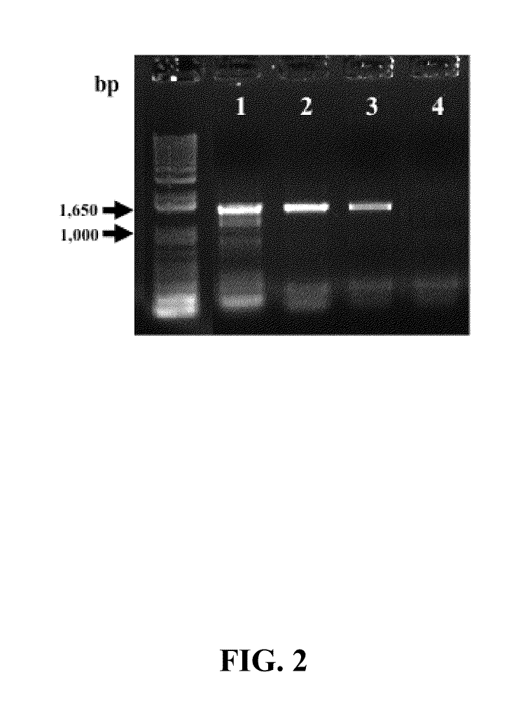

To obtain the missing 5′ end of the RDase A gene from the Dhc mccartyi RC and KS strains (dcpA), an additional PCR primer (dcp_up120F; 5′-GCTCCTGGCAGAGCCGTCAGT-3′; SEQ ID NO: 15) targeting a region upstream of the dcpA gene was designed based on the genome of Dehalogenimonas lykanthroporepellens strain BL-DC-9 (NCBI accession number NC_{—}014314). The dcp_up120F primer (SEQ ID NO: 15) was designed in view of the sequence similarity between the dcpAB gene in the RC and KS strains and the RDase gene from Dehalogenimonas lykanthroporepellens strain BL-DC-9, to target an intergenic region around 120 by upstream of the dcpA gene start position. This primer, in combination with the dcpA-1449R primer (5′-TTTAAACAGCGGGCAGGTACTGGT-3′; SEQ ID NO: 16), was used in PCR reactions using Dhc mccartyi strain RC and strain KS genomic DNA. The PCR reactions consisted of (final concentrations) PCR buffer (1×), 2.5 mM of MgCl₂, 250 μM of each deoxynucleoside triphosphate (ABI), primers (250 nM each), and 2.5 U of AmpliTaq polymerase (ABI). The following thermocycler temperature program for the amplification of the dcpA gene was used: 94° C. for 2 min, 10 seconds (1 cycle); 94° C. for 30 seconds, 56.0° C. for 45 seconds, and 72° C. for 2 min, 10 seconds (30 cycles); and 72° C. for 6 min. The PCR products were run on a 1% (wt/vol) agarose gel and stained with ethidium bromide (1 μg/mL) as shown in FIG. 2. A PCR product of the expected size (˜1,569 bp) was obtained using Dhc mccartyi strain RC and strain KS genomic DNA as a template (see lanes 2-3, respectively, of FIG. 2). Genomic DNA from Dehalogenimonas lykanthroporepellens strain BL-DC-9 served as a positive control (see lane 1).

The PCR product was purified with the Qiagen PCR Purification kit (Qiagen, Valencia, Calif., USA) and sequenced. Alignments of sequences using SeqMan II software (DNASTAR, Lasergene version 7) and ClustalW were used to assemble the missing 5′ end and dcpA start coding sequence. In addition, the DNA Walking SpeedUp™ (Seegene, Korea) kit was used to extend the partial dcpAB sequence to amplify the whole sequence of the RDase B gene. The procedure involved a series of consecutive PCR amplifications with primers targeting known sequence regions in combination with the kit's DNA Walking Annealing Control Primers. The internal primers dcpA-360F (5′-TTGCGTGATCAAATTGGAGCCTGG-3′; SEQ ID NO: 17) and dcpA-1257F (5′-CGATGTGCCAGCCATTGTGTCTTT-3′; SEQ ID NO: 18) were used with Dhc mccartyi strain RC and strain KS genomic DNA following the DNA Walking kit manufacturer's recommendations. The resulting chromosome walking PCR products were purified, sequenced and assembled as described above.

The coding DNA sequence of the dcpA gene from the Dhc mccartyi strain KS is provided as SEQ ID NO: 1, and the sequence is set forth below:

>Dhc_KS_dcpA
ATGAAATCGCATTCCACAATGAGTCGCCGAGATTTTATGAAATCCATAGG
TTTAGGTTCCGCAGCCATAGCTAGCATGGGTGCAACCGCTCCCTTTTTCC
ATGATCTAGACGAAATGACAGGCATAGGTGCTGCCGAAACTTTTAATTCC
ACGACATCTATGCAAAAACGTCCTTGGTGGGTTAAAGAAGTTGACATTCC
AACAGTAGAAATCGACTTGAAACTACGCACGCCTTACGCCGGCCCTACGC
CATTAGCTGGAACATTAGCCTCAATATATGTTACCAAAGAAGAGACTGCT
GCTATCCTGGCTTCCCAAAAGAACAATGCCATTGAAGGAGCCAAAAACAA
CCGGCCAGGTTTTACATTGCGTGATCAAATTGGAGCCTGGGCCTCGTTAG
ATAGAGGACAAACGGGATATGTGAAATATCCGCCTGAAGGTTTTCGAACA
ATTAAAGTCACCCATGAAACTTTAGGTGTACCGAAGTGGGAAGGCTCCGA
AACGGAAAACGCATTCATGATTCGAACTTTCCTGAGGCAATTTGGTGCAG
GGGCGATTGGCTATGCAAGAGTGGATGACGACAGCGTCGGACCACGTAAA
CCCCTTTTCAATACACATGTGAGATTGGAAAACAACGCAGATTATAAGTA
CGATTCTAATGGCGTATTTGTCATGCCAGAAAAATGCAAGTATGCCATTA
TTATGTATGACAGAAGTCCCCGAGATCCTAACAACTATCGTCGTACTGTG
AATAGCCCTCAAGCTTTTGTATCAAACATGGAAAAATGTGAGTATGGTCA
TAAGCTTCAAAACTTCCTTTGGGGCTTAGGCTACCAGTCTTATTGGTTTG
AAGACGGTACAACTAGTAAGTTTACTGGGACCCCGACTAATGTTTGGGGT
ATTCTCTCAGGTGTAGGAGAGTATAACCGAATTCACAATGCTGTTTCACA
ACCAGAAGGCGAGAGCGGCAATTTTGCAAGTATTCTCTTTACCGATTTAC
CTTTGCCCACAACTAAACCTATAGACTTTGGTGCCTTGGAATTCTGTAAA
ACTTGTGGGATATGTGCCGACGTTTGCCCAGCCGGAGCAATTCCTACAGT
AGAAGAATATCGAGAGCCAACTTGGGATCGAGCAACTGGTCCCTGGAGTG
CTTCCAATGACCATAAAGGATATCCTAATAAATCCATTGAATGCGTAAAA
TGGTATTTTTCCTATGCAATTACAGCGTACGCCCCTTCATCTCGCCCAGT
TGGTGTGTGTCGTCGATGTGCCAGCCATTGTGTCTTTAGTAAAGATCATG
AAGCTTGGATTCATGAAGTAGTTAAGGGTGTAGTTTCCACTACCCCTGTG
ATGAACAGCTTCTTTACTAAAATGGATATGCTATCCGGTTACAGTGACGT
CATCTCAGATGAAGGCAGAGCTGAATATTGGCACCAGTACCTGCCCGCTG
TTTAA

The amino acid sequence of the DcpA protein from the Dhc mccartyi strain KS, as determined from the corresponding DNA sequence, is provided as SEQ ID NO: 2, and the sequence is set forth below using the standard one-letter designations for the amino acids:

>Dhc_KS_DcpA
MKSHSTMSRRDFMKSIGLGSAAIASMGATAPFFHDLDEMTGIGAAETFNS
TTSMQKRPWWVKEVDIPTVEIDLKLRTPYAGPTPLAGTLASIYVTKEETA
AILASQKNNAIEGAKNNRPGFTLRDQIGAWASLDRGQTGYVKYPPEGFRT
IKVTHETLGVPKWEGSETENAFMIRTFLRQFGAGAIGYARVDDDSVGPRK
PLFNTHVRLENNADYKYDSNGVFVMPEKCKYAIIMYDRSPRDPNNYRRTV
NSPQAFVSNMEKCEYGHKLQNFLWGLGYQSYWFEDGTTSKFTGTPTNVWG
ILSGVGEYNRIHNAVSQPEGESGNFASILFTDLPLPTTKPIDFGALEFCK
TCGICADVCPAGAIPTVEEYREPTWDRATGPWSASNDHKGYPNKSIECVK
WYFSYAITAYAPSSRPVGVCRRCASHCVFSKDHEAWIHEVVKGVVSTTPV
MNSFFTKMDMLSGYSDVISDEGRAEYWHQYLPAV

The coding DNA sequence of the dcpA gene from the Dhc mccartyi strain RC is provided as SEQ ID NO: 3, and the sequence is set forth below:

>Dhc_RC_dcpA
ATGAAATCGCATTCCACAATGAGTCGCCGAGATTTTATGAAATCCATAGG
TTTAGGTTCCGCAGCCATAGCTAGCATGGGTGCAACCGCTCCCTTTTTCC
ATGATCTAGACGAAATGACAGGCATAGGTGCTGCCGAAACTTTTAATTCC
ACGACATCTATGCAAAAACGTCCTTGGTGGGTTAAAGAAGTTGACATTCC
AACAGTAGAAATCGACTTGAAACTACGCACGCCTTACGCCGGCCCTACGC
CATCAGCTGGAACATTAGCCTCAATATATGTTACCAAAGAAGAGACTGCT
GCTATCCTGGCTTCCCAAAAGAACAATGCCATTGAAGGAGCCAAAAACAA
CCGGCCAGGTTTTACATTGCGTGATCAAATTGGAGCCTGGGCCTCGTTAG
ATAGAGGACAAACGGGATATGTGAAATATCCGCCTGAAGGTTTTCGAACA
ATTAAAGTCACCCATGAAACTTTAGGTGTACCGAAGTGGGAAGGCTCCGA
AACGGAAAACGCATTCATGATTCGAACTTTCCTGAGGCAATTTGGTGCAG
GGGCGATTGGCTATGCAAGAGTGGATGACGACAGCGTCGGACCACGTAAA
CCCCTTTTCAATACACATGTGAGATTGGAAAACAACGCAGATTATAAGTA
CGATTCTAATGGCGTATTTGTCATGCCAGAAAAATGCAAGTATGCCATTA
TTATGTATGACAGAAGTCCCCGAGATCCTAACAACTATCGTCGTACTGTG
AATAGCCCTCAAGCTTTTGTATCAAACATGGAAAAATGTGAGTATGGTCA
TAAGCTTCAAAACTTCCTTTGGGGCTTAGGCTACCAGTCTTATTGGTTTG
AAGACGGTACAACTAGTAAGTTTACTGGGACCCCGACTAATGTTTGGGGT
ATTCTCTCAGGTGTAGGAGAGTATAACCGAATTCACAATGCTGTTTCACA
ACCAGAAGGCGAGAGCGGCAATTTTGCAAGTATTCTCTTTACCGATTTAC
CTTTGCCCACAACTAAACCTATAGACTTTGGTGCCTTGGAATTCTGTAAA
ACTTGTGGGATATGTGCCGACGTTTGCCCAGCCGGAGCAATTCCTACAGT
AGAAGAATATCGAGAGCCAACTTGGGATCGAGCAACTGGTCCCTGGAGTG
CTTCCAATGACCATAAAGGATATCCTAATAAATCCATTGAATGCGTAAAA
TGGTATTTTTCCTATGCAATTACAGCGTACGCCCCTTCATCTCGCCCAGT
TGGTGTGTGTCGTCGATGTGCCAGCCATTGTGTCTTTAGTAAAGATCATG
AAGCTTGGATTCATGAAGTAGTTAAGGGTGTAGTTTCCACTACCCCTGTG
ATGAACAGCTTCTTTACTAAAATGGATATGCTATCCGGTTACAGTGACGT
CATCTCAGATGAAGGCAGAGCTGAATATTGGCACCAGTACCTGCCCGCTG
TTTAA

The amino acid sequence of the DcpA protein from the Dhc mccartyi strain RC, as determined from the corresponding DNA sequence, is provided as SEQ ID NO: 4, and the sequence is set forth below:

>Dhc_RC_DcpA
MKSHSTMSRRDFMKSIGLGSAAIASMGATAPFFHDLDEMTGIGAAETFNS
TTSMQKRPWWVKEVDIPTVEIDLKLRTPYAGPTPSAGTLASIYVTKEETA
AILASQKNNAIEGAKNNRPGFTLRDQIGAWASLDRGQTGYVKYPPEGFRT
IKVTHETLGVPKWEGSETENAFMIRTFLRQFGAGAIGYARVDDDSVGPRK
PLFNTHVRLENNADYKYDSNGVFVMPEKCKYAIIMYDRSPRDPNNYRRTV
NSPQAFVSNMEKCEYGHKLQNFLWGLGYQSYWFEDGTTSKFTGTPTNVWG
ILSGVGEYNRIHNAVSQPEGESGNFASILFTDLPLPTTKPIDFGALEFCK
TCGICADVCPAGAIPTVEEYREPTWDRATGPWSASNDHKGYPNKSIECVK
WYFSYAITAYAPSSRPVGVCRRCASHCVFSKDHEAWIHEVVKGVVSTTPV
MNSFFTKMDMLSGYSDVISDEGRAEYWHQYLPAV

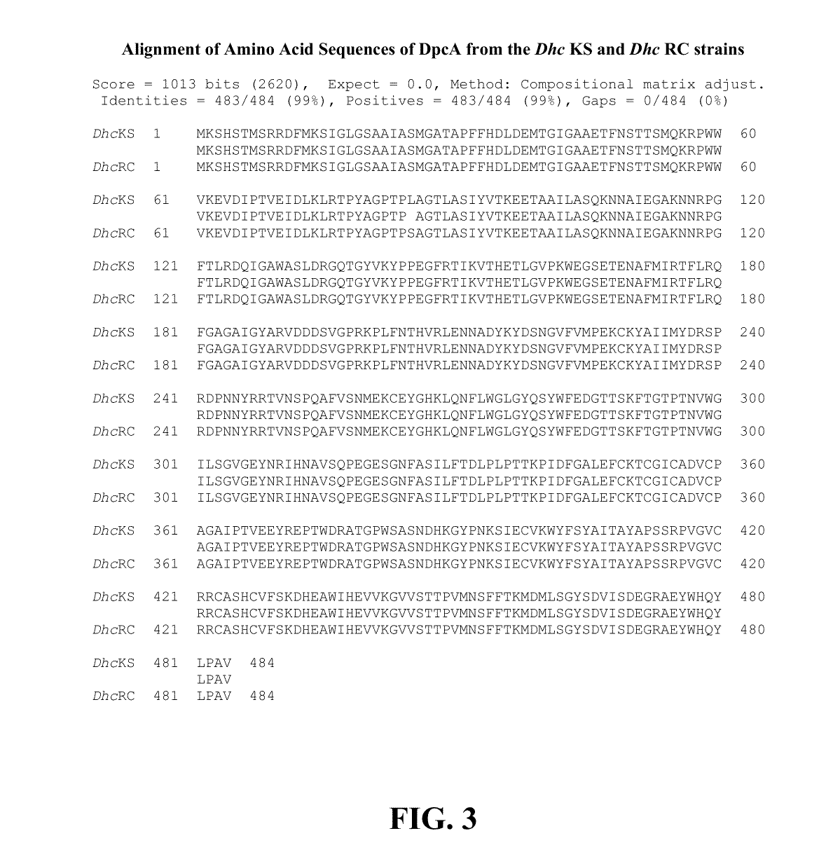

FIG. 3 is an amino acid sequence alignment of the amino acid sequence of the DcpA protein from the Dhc mccartyi KS strain (denoted DhcKS in FIG. 3) and the amino acid sequence of the DcpA protein from the Dhc mccartyi RC strain (denoted DhcRC in FIG. 3) that was made using the Align Sequences Protein BLAST tool (default parameters) available from the National Center for Biotechnology Information (NCBI) website at http://blast.ncbi.nlm.nih.gov/Blast.cgi?PAGE=Proteins&PROGRAM=blastp&BLAST_PROGRAMS=blastp&PAGE_TYPE=BlastSearch&BLAST_SPEC=blast2seq. As can be seen in FIG. 3, the amino acid sequences of the DcpA proteins from the Dhc mccartyi KS and RC strains are greater than 99% identical, with only one amino acid in the 484-amino acid sequences differing between the KS and RC strains. In particular, the leucine (L) at position 85 of the Dhc mccartyi strain KS DcpA protein is a serine (S) in the Dhc mccartyi strain RC DcpA protein.

The coding DNA sequence of the dcpB gene from the Dhc mccartyi KS strain is provided as SEQ ID NO: 5, and the sequence is set forth below:

>dcpB_KS
ATGAATTTTAATATTGACTTTAAATGGTATCAATGGCTTTTTGGAGTGAT
TTCTCTTATTCTGGCATCTTTCTTGACTCATGAAGTATTTGCTACCCTAG
CAGAATCTCAGCCTGGCACTGTCAAGGTACTTTCCTTGCTTATAGGAATC
CCATTGATTATCTTCCTGTATCTGACCTTTGGTTTAAGGTCGGCCTTAAA
AAAACACAAGTCTAACTAA

The amino acid sequence of the DcpB protein from the Dhc mccartyi KS strain, as determined from the corresponding DNA sequence, is provided as SEQ ID NO: 6, and the sequence is set forth below:

>DcpB_KS
MNFNIDFKWYQWLFGVISLILASFLTHEVFATLAESQPGTVKVLSLLIGI
PLIIFLYLTFGLRSALKKHKSN

The coding DNA sequence of the dcpB gene from the Dhc mccartyi RC strain is provided as SEQ ID NO: 7, and the sequence is set forth below:

>dcpB_RC
ATGAATTTTAATATTGACTTTAAATGGTATGAATGGCTTTTTGGAGTGAT
TTCTCTTATTCTGGCATCTTTCTTGACTCATGAAGTATTTGCTACCCTAG
CAGAATCTCAGCCTGGCACTGTCAAGGTACTTTCCTTGCTTATAGGAATC
CCATTGATTATCTTCCTGTATCTGACCTTTGGTTTAAGGTCGGCCTTAAA
AAAACACAAGTCTAACTAA

The amino acid sequence of the DcpB protein from the Dhc mccartyi RC strain, as determined from the corresponding DNA sequence, is provided as SEQ ID NO: 8, and the sequence is set forth below:

>DcpB_RC
MNFNIDFKWYEWLFGVISLILASFLTHEVFATLAESQPGTVKVLSLLIGI
PLIIFLYLTFGLRSALKKHKSN

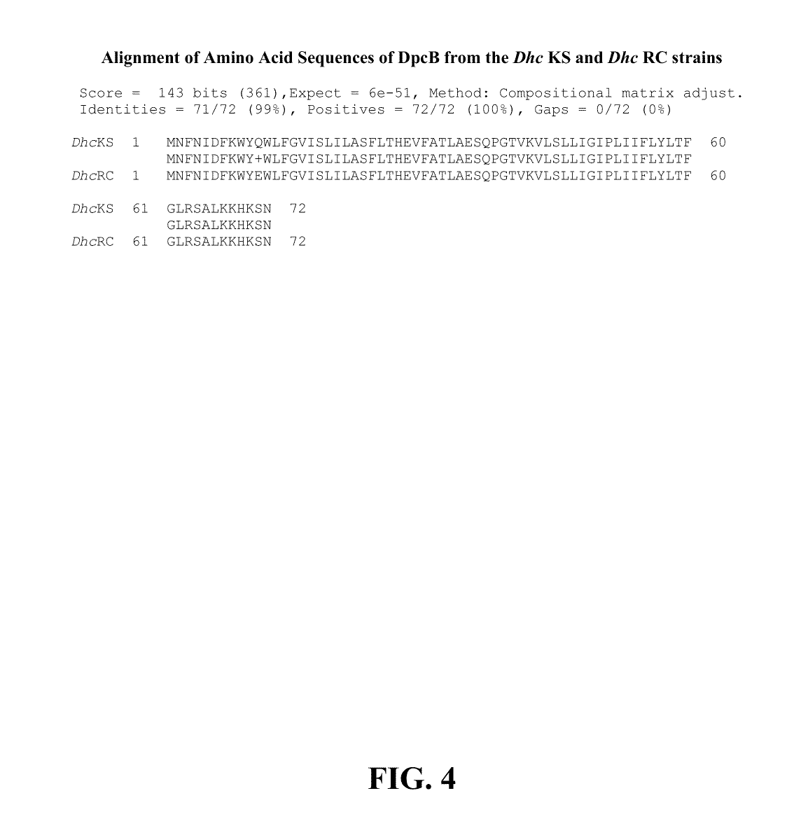

FIG. 4 is an amino acid sequence alignment of the amino acid sequence of the DcpB protein from the Dhc mccartyi KS strain (denoted DhcKS in FIG. 4) and the amino acid sequence of the DcpB protein from the Dhc mccartyi RC strain (denoted DhcRC in FIG. 4) that was made using the Align Sequences Protein BLAST tool (default parameters) available from the National Center for Biotechnology Information (NCBI) website at http://blast.ncbi.nlm.nih.gov/Blast.cgi?PAGE=Proteins&PROGRAM=blastp&BLAST_PROGRAMS=blastp&PAGE_TYPE=BlastSearch&BLAST_SPEC=blast2seq. As can be seen in FIG. 4, the amino acid sequences of the DcpB proteins from the Dhc mccartyi KS and RC strains are greater than 99% identical, with only one amino acid in the 73-amino acid sequences differing between the KS and RC strains. In particular, the glutamine (Q) at position 11 of the Dhc mccartyi strain KS DcpB protein is a glutamic acid (E) in the Dhc mccartyi strain RC DcpB protein.

### Example 5

### Sequence Analysis of the dcpA and dcpB Genes and the DcpA and DcpB Proteins

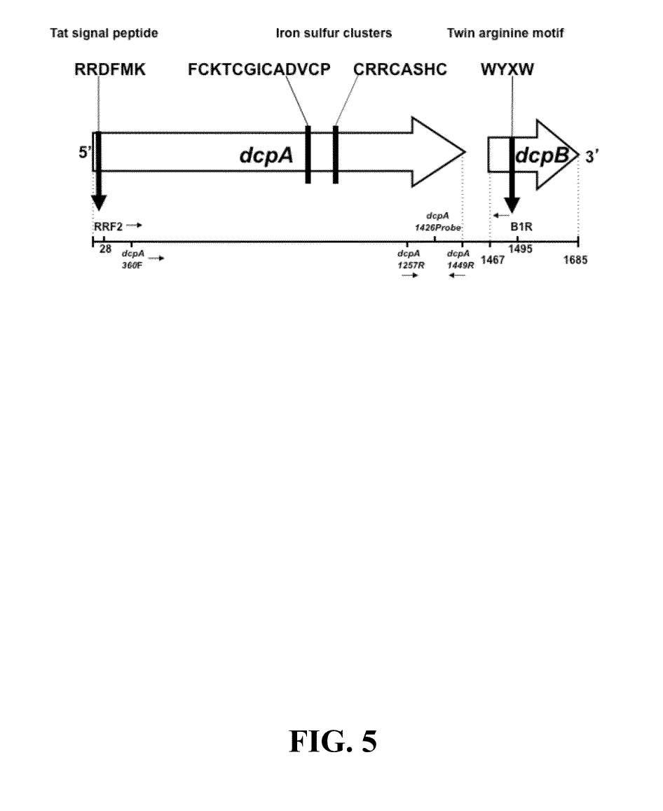

The DNA sequences of the dcpA and dcpB genes and the corresponding amino acid sequences of the DcpA and DcpB proteins were analyzed, and some of the identified features are shown schematically in FIG. 5. For example, the DcpA amino acid sequences were analyzed for secretory signal peptides using the TatP (www.cbs.dtu.dk/services/TatP) and SignalP (http://www.cbs.dtu.dk/services/SignalP/) programs. The “Compute pI/Mw” program (http://us.expasy.org/tools/pi_tool.html) was used to predict the molecular weight and isoelectric point of DcpA. The DcpB sequences were analyzed with TMMOD (http://www.liao.cis.udel.edu/website/servers/TMMOD/), a web-based program that uses a Hidden Markov model to predict protein topology based on transmembrane motifs.

Both the RC and KS DcpA amino acid sequences have a Tat signal peptide with the sequence RRDFMK (see FIG. 5) starting at amino acid position nine and a predicted peptide cleavage site between amino acid positions 30 and 31. The mature DcpA protein (after cleavage of the signal peptide) in the Dhc mccartyi RC and KS strains has a theoretical isoelectric point (pI) of 5.99 and a molecular weight (MW) of about 50.8 kDa, which is comparable to other RDases. Additionally, the DcpA proteins identified in the Dhc mccartyi RC and KS strains have two iron sulfurs clusters (a characteristic of RDases) in the form FCX₂CX₂CX₃CP and CX₂CX₃C, as illustrated in FIG. 5. The cobalamin-binding motif DXHXXG-x₄₁₋₄₂-SXL-x₂₄₋₂₈-GG found in various corrinoid-containing enzymes does not appear to be present in DcpA but a variant putative cobalamin-binding sequence close to the C-terminal end in the form DHKG-X₅-SIE-X₁₉-G was identified.

As shown in FIG. 5, the conserved motif WYXW present in other RDase B proteins is present as WYEW in the Dhc mccartyi RC strain and as WYQW in the Dhc mccartyi KS strain. Both the RC and KS DcpB amino acid sequences have two predicted transmembrane regions stretching from amino acid positions 12-32 and 41-61. The remainder of the deduced topology shows that DcpB has two inside loops from amino acid positions 1-11 and 62-73 and one outside loop from positions 33-40. Furthermore, a putative RBS site (GAGG) for the dcpB gene was detected in the small intergenic region between dcpA and dcpB.

DNA sequences that include both the dcpA and dcpB genes from the Dhc mccartyi KS and RC strains are provided as SEQ ID NO: 9 and SEQ ID NO: 10, respectively:

dcpAB gene from Dhc KS strain, SEQ ID NO: 9:
GTGTCTTGTCAGCCTCCCTGTGTTCATTCTAGTAAACAAGGGTTACAGAG
TCAAGGAACCAACAAAAATCAAAACATAAAAGAGGTAATATGAAATCGCA
TTCCACAATGAGTCGCCGAGATTTTATGAAATCCATAGGTTTAGGTTCCG
CAGCCATAGCTAGCATGGGTGCAACCGCTCCCTTTTTCCATGATCTAGAC
GAAATGACAGGCATAGGTGCTGCCGAAACTTTTAATTCCACGACATCTAT
GCAAAAACGTCCTTGGTGGGTTAAAGAAGTTGACATTCCAACAGTAGAAA
TCGACTTGAAACTACGCACGCCTTACGCCGGCCCTACGCCATTAGCTGGA
ACATTAGCCTCAATATATGTTACCAAAGAAGAGACTGCTGCTATCCTGGC
TTCCCAAAAGAACAATGCCATTGAAGGAGCCAAAAACAACCGGCCAGGTT
TTACATTGCGTGATCAAATTGGAGCCTGGGCCTCGTTAGATAGAGGACAA
ACGGGATATGTGAAATATCCGCCTGAAGGTTTTCGAACAATTAAAGTCAC
CCATGAAACTTTAGGTGTACCGAAGTGGGAAGGCTCCGAAACGGAAAACG
CATTCATGATTCGAACTTTCCTGAGGCAATTTGGTGCAGGGGCGATTGGC
TATGCAAGAGTGGATGACGACAGCGTCGGACCACGTAAACCCCTTTTCAA
TACACATGTGAGATTGGAAAACAACGCAGATTATAAGTACGATTCTAATG
GCGTATTTGTCATGCCAGAAAAATGCAAGTATGCCATTATTATGTATGAC
AGAAGTCCCCGAGATCCTAACAACTATCGTCGTACTGTGAATAGCCCTCA
AGCTTTTGTATCAAACATGGAAAAATGTGAGTATGGTCATAAGCTTCAAA
ACTTCCTTTGGGGCTTAGGCTACCAGTCTTATTGGTTTGAAGACGGTACA
ACTAGTAAGTTTACTGGGACCCCGACTAATGTTTGGGGTATTCTCTCAGG
TGTAGGAGAGTATAACCGAATTCACAATGCTGTTTCACAACCAGAAGGCG
AGAGCGGCAATTTTGCAAGTATTCTCTTTACCGATTTACCTTTGCCCACA
ACTAAACCTATAGACTTTGGTGCCTTGGAATTCTGTAAAACTTGTGGGAT
ATGTGCCGACGTTTGCCCAGCCGGAGCAATTCCTACAGTAGAAGAATATC
GAGAGCCAACTTGGGATCGAGCAACTGGTCCCTGGAGTGCTTCCAATGAC
CATAAAGGATATCCTAATAAATCCATTGAATGCGTAAAATGGTATTTTTC
CTATGCAATTACAGCGTACGCCCCTTCATCTCGCCCAGTTGGTGTGTGTC
GTCGATGTGCCAGCCATTGTGTCTTTAGTAAAGATCATGAAGCTTGGATT
CATGAAGTAGTTAAGGGTGTAGTTTCCACTACCCCTGTGATGAACAGCTT
CTTTACTAAAATGGATATGCTATCCGGTTACAGTGACGTCATCTCAGATG
AAGGCAGAGCTGAATATTGGCACCAGTACCTGCCCGCTGTTTAAAATGAG
AGAGGAAAAAACATGAATTTTAATATTGACTTTAAATGGTATCAATGGCT
TTTTGGAGTGATTTCTCTTATTCTGGCATCTTTCTTGACTCATGAAGTAT
TTGCTACCCTAGCAGAATCTCAGCCTGGCACTGTCAAGGTACTTTCCTTG
CTTATAGGAATCCCATTG
dcpAB gene from Dhc RCstrain, SEQ ID NO: 10:
GTGTCTTGTCAGCCTCCCTGTGTTCATTCTAGTAAACAAGGGTTACAGAG
TCAAGGAACCAACAAAAATCAAAACATAAAAGAGGTAATATGAAATCGCA
TTCCACAATGAGTCGCCGAGATTTTATGAAATCCATAGGTTTAGGTTCCG
CAGCCATAGCTAGCATGGGTGCAACCGCTCCCTTTTTCCATGATCTAGAC
GAAATGACAGGCATAGGTGCTGCCGAAACTTTTAATTCCACGACATCTAT
GCAAAAACGTCCTTGGTGGGTTAAAGAAGTTGACATTCCAACAGTAGAAA
TCGACTTGAAACTACGCACGCCTTACGCCGGCCCTACGCCATCAGCTGGA
ACATTAGCCTCAATATATGTTACCAAAGAAGAGACTGCTGCTATCCTGGC
TTCCCAAAAGAACAATGCCATTGAAGGAGCCAAAAACAACCGGCCAGGTT
TTACATTGCGTGATCAAATTGGAGCCTGGGCCTCGTTAGATAGAGGACAA
ACGGGATATGTGAAATATCCGCCTGAAGGTTTTCGAACAATTAAAGTCAC
CCATGAAACTTTAGGTGTACCGAAGTGGGAAGGCTCCGAAACGGAAAACG
CATTCATGATTCGAACTTTCCTGAGGCAATTTGGTGCAGGGGCGATTGGC
TATGCAAGAGTGGATGACGACAGCGTCGGACCACGTAAACCCCTTTTCAA
TACACATGTGAGATTGGAAAACAACGCAGATTATAAGTACGATTCTAATG
GCGTATTTGTCATGCCAGAAAAATGCAAGTATGCCATTATTATGTATGAC
AGAAGTCCCCGAGATCCTAACAACTATCGTCGTACTGTGAATAGCCCTCA
AGCTTTTGTATCAAACATGGAAAAATGTGAGTATGGTCATAAGCTTCAAA
ACTTCCTTTGGGGCTTAGGCTACCAGTCTTATTGGTTTGAAGACGGTACA
ACTAGTAAGTTTACTGGGACCCCGACTAATGTTTGGGGTATTCTCTCAGG
TGTAGGAGAGTATAACCGAATTCACAATGCTGTTTCACAACCAGAAGGCG
AGAGCGGCAATTTTGCAAGTATTCTCTTTACCGATTTACCTTTGCCCACA
ACTAAACCTATAGACTTTGGTGCCTTGGAATTCTGTAAAACTTGTGGGAT
ATGTGCCGACGTTTGCCCAGCCGGAGCAATTCCTACAGTAGAAGAATATC
GAGAGCCAACTTGGGATCGAGCAACTGGTCCCTGGAGTGCTTCCAATGAC
CATAAAGGATATCCTAATAAATCCATTGAATGCGTAAAATGGTATTTTTC
CTATGCAATTACAGCGTACGCCCCTTCATCTCGCCCAGTTGGTGTGTGTC
GTCGATGTGCCAGCCATTGTGTCTTTAGTAAAGATCATGAAGCTTGGATT
CATGAAGTAGTTAAGGGTGTAGTTTCCACTACCCCTGTGATGAACAGCTT
CTTTACTAAAATGGATATGCTATCCGGTTACAGTGACGTCATCTCAGATG
AAGGCAGAGCTGAATATTGGCACCAGTACCTGCCCGCTGTTTAAAATGAG
AGAGGAAAAAACATGAATTTTAATATTGACTTTAAATGGTATGAATGGCT
TTTTGGAGTGATTTCTCTTATTCTGGCATCTTTCTTGACTCATGAAGTAT
TTGCTACCCTAGCAGAATCTCAGCCTGGCACTGTCAAGGTACTTTCCTTG
CTTATAGGAATCCCATTGATTATCTTCCTGTATCTGACCTTTGGTTTAAG
GTCGGCCTTAAAAAAACACAAGTCTAACTAA

Inspection of the upstream region of the dcpA genes (before the start codon) identifies a putative ribosome-binding site (RBS or Shine-Dalgarno sequence) with the sequence AGAGG that is nine nucleotides upstream of the dcpA start codon. Furthermore, a Pribnow box (or the −10 element) with the sequence TAATAT and a putative Dehalobox were identified upstream of the dcpA start codon. The Pribnow box is a consensus sequence in the promoter region of a gene that is recognized by the RNA polymerase and Dehaloboxes consist of a stretch of nucleotides that resemble the FNR-box that binds to the promoter and induces transcription of genes involved in reductive dechlorination (Pribnow, D., et al. (1975) Proc. Nat. Acad. Sci. USA. 72:784-788; Smidt, H., et al. (2000) J. Bacteriol. 182:5683-5691; Gabor, K., et al. (2006) J. Bacteriol. 188:2604-2613).

### Example 6

### Quantitative Real-Time PCR Assays

For the dcpA quantitative PCR approach, the recommended strategy outlined by a recent study (Hatt J. K., and F. E. Löffler (2011) J. Microbiol. Methods. 88:263-270) was used. In summary, the primers were in-silico designed for TaqMan assays, the primers and the PCR conditions were first tested with SYBR Green chemistry (i.e., to check for non-specific amplification and/or primer-dimer formation), and finally the optimized PCR conditions were used with the TaqMan probe in TaqMan-based assays.

The Quantitative Real-Time PCR (qPCR) probe and primers directed to the dcpA gene were designed using the IDT DNA Primer Quest software (http://scitools.idtdna.com/Primerquest/) using the software's default parameters. The following Quantitative Real-Time PCR primers and probe were selected for the quantification of the dcpA gene: the dcpA-1257F primer (5′-CGATGTGCCAGCCATTGTGTCTTT-3′; SEQ ID NO: 18), the dcpA-1449R primer (5′-TTTAAACAGCGGGCAGGTACTGGT-3′; SEQ ID NO: 16) and the dcpA-1426Probe sequence (5′-6FAM-ACGTCATCTCAGATGAAGGCAGAGCT-3′-BHQ (Black Hole Quencher); SEQ ID NO: 20).

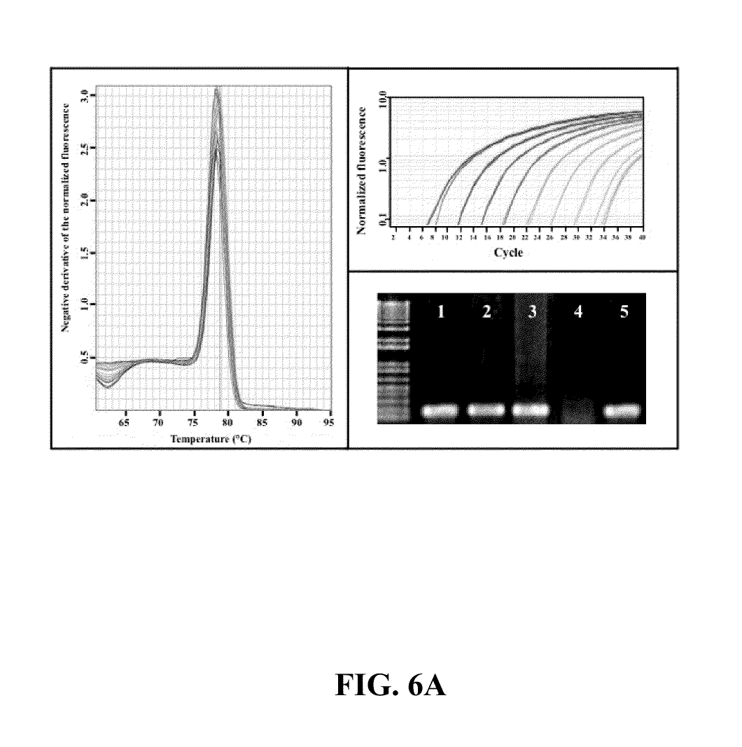

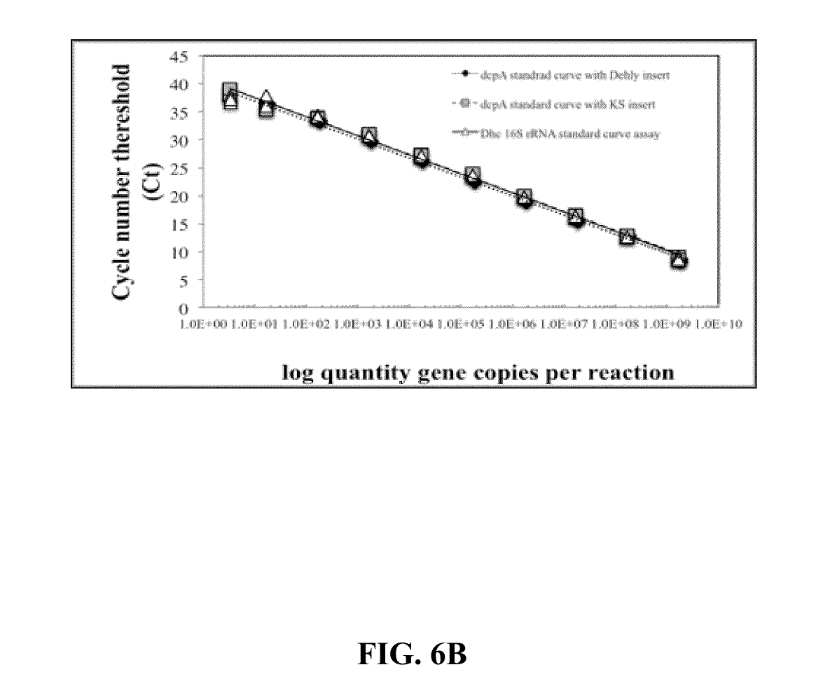

To evaluate primer specificity and amplification efficiency, the Power SYBR Green PCR master mix (ABI; product no. 4367659) was used in reaction volumes of 20 μL. The reactions consisted of 1× master mix (ABI), 300 nM each of reverse and forward primers, and 2 μL of template DNA. At the end of each cycle, a melting curve analysis from 67° C. to 95° C. was performed using the ABI default settings. Standard curves were generated with ten-fold dilutions of the partial Dhc mccartyi strain KS dcpA gene fragment cloned in the TOPO TA (Invitrogen) pCRII plasmid. Each dilution was run in triplicate. To confirm target specificity, melting curves were generated with SYBR Green chemistry using genomic DNA from Dhc mccartyi strains RC and KS and Dehalogenimonas lykanthroporepellens strain BL-DC-9. The melting curve analysis showed a single sharp peak annealing (indicating a single PCR product) with an average melting temperature (Tₘ) of 78.8±0.4, affirming the specificity of the primers and the absence of primer-dimer formation in the assay (see FIG. 6A). Reactions with only water (no-template) and with genomic DNA from Dhc strain GT (a strain that does not possess the dcpA gene) were used as negative controls and no amplification was seen in these negative controls. After validating the Quantitative Real-Time PCR primers and reaction conditions with the SYBR Green chemistry, TaqMan-based assays were tested.

TaqMan-based assays were successfully developed for the quantification of dcpA. The dcpA TaqMan assay consisted of 1×TaqMan Universal PCR master mix (ABI; product no. 4304437), 300 nM of probe, 300 nM each of reverse and forward primers and 2 μL of purified DNA and/or diluted cDNA (1:50 and 1:100) as template in a total volume of 20 μL. The PCR cycle parameters were as follows: 2 min at 50° C. (to activate the AmpErase UNG present in the master mix used to minimize the contamination of previous reaction products), denaturation for 10 min at 95° C., followed by 40 cycles of 15 seconds at 95° C. and 1 min at 60° C. Gene copies per reaction or per μL of template DNA in standards or unknown samples were calculated as previously described (Ritalahti, K. M., et al. (2006) Appl. Environ. Microbiol. 72:2765-2774). The numbers of target genes were normalized to milliliters of culture or grams of solids. For all assays standard curves were run in every plate and an amplification threshold value of 0.2 was set. All qPCR assays complied with optimal conditions, having an amplification efficiency between 90-100% with a slope within −3.6 and −3.1. (Bustin, S. A., et al. (2009). Clinical Chemistry 55:611-22). The detection limit of the dcpA TaqMan qPCR assays with primers dcpA-1257F (SEQ ID NO: 18), dcpA-1449R (SEQ ID NO: 16) and probe dcpA-1426Probe (SEQ ID NO: 20) was considered the lowest value for the linear calibration curve that exhibited linear amplification in the range of 10⁰ to 10⁹ dcpA gene copies per of template. Amplification efficiencies and slopes were comparable to standard curves of Dhc 16S rRNA qPCR assays (see FIGS. 6A and 6B). No difference in amplification efficiency and quantification was seen when the Dehalococcoides dcpA gene or Dehalogenimonas dcpA gene inserts were used to generate standards curves. Five μL of the resulting qPCR products were mix with 1 μL of 6× loading dye and resolved in 1% (wt/vol) agarose gels and stained with ethidium bromide (1 μg/ml); the resulting gel image confirmed the amplification of single amplicons of the expected size (see FIG. 6A).

### Example 7

### PCR Reactions Directed to dcpA Gene

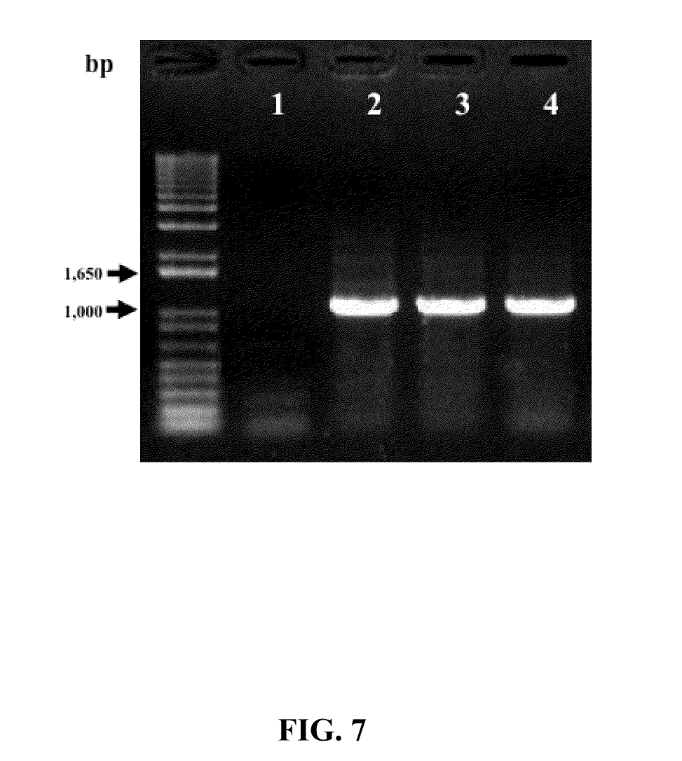

Additional Primers were Also Developed that were Directed to the dcpA Genes. Primers dcpA-360F (TTGCGTGATCAAATTGGAGCCTGG; SEQ ID NO: 17) and dcpA-1449R (TTTAAACAGCGGGCAGGTACTGGT; SEQ ID NO: 16) were designed based on alignments of the dcpA sequences retrieved from the cDNA library of Dehalococcoides mccartyi strains RC and KS as well as the dcpA gene (Dehly_{—}1524) detected in the Dehalogenimonas genome. Direct and nested PCR with genomic DNA of Dhc mccartyi strain RC and strain KS cultures produced a single amplicon of the expected size (about 1,089 bp) as shown in FIG. 7. Lanes 2-4 of FIG. 7 correspond to PCR amplicons using primers dcpA-360F (SEQ ID NO: 17) and dcpA-1449R (SEQ ID NO: 16) and 10 ng of genomic DNA from the Dhc mccartyi strain RC, Dhc mccartyi strain KS and Dehalogenimonas lykanthroporepellens (Dhg) strain BL-DC-9, respectively. Lane 1 of the agarose gel in FIG. 1 is a PCR control with no template DNA. A 1 kb Plus DNA Ladder (Invitrogen) is included in the left-most lane of the agarose gel.

Experiments to determine the detection limit of the direct PCR approach using serially diluted plasmid DNA with the dcpAB insert showed that only 17 gene copies per μL of template DNA were needed to produce amplicons that generated a visible band following ethidium bromide-staining in 1% (wt/vol) agarose gels for direct PCR. The detection limit of the dcpA direct PCR assay was tested by performing PCR with 10-fold serial dilutions of plasmid DNA containing the partial dcpAB gene of Dhc mccartyi strain KS. The PCR reactions consisted of (final concentrations) PCR buffer (1×), 2.5 mM of MgCl₂, 250 μM of each deoxynucleoside triphosphate (ABI), primers dcpA-360F (SEQ ID NO: 17) and dcpA-1449R (SEQ ID NO: 16) (250 nM each), 2 μL of serially diluted plasmid (1.7×10⁸-1.7×10⁰ per μL of template DNA), and 2.5 U of AmpliTaq polymerase (ABI). The following thermocycler temperature program for the amplification of the dcpA gene was used: 94° C. for 2 min, 10 seconds (1 cycle); 94° C. for 30 seconds, 56.0° C. for 45 seconds, and 72° C. for 2 min, 10 seconds (30 cycles); and 72° C. for 6 min. Five μL of PCR product were mixed with 1 μL of 6× loading dye and resolved in 1% (wt/vol) agarose gels and stained with ethidium bromide (1 μg/mL). The detection limit was determined as the lowest dilution that yielded a visible amplicon band in an ethidium bromide stained gel. As discussed above, at least 17 template DNA copies per μL were required to obtain a visible band. To explore the effects of environmental DNA on the detection sensitivity in direct PCR reactions, the same experiment was performed but with the modification of adding 10 ng of exogenous groundwater DNA to each reaction tube. The sensitivity of the assay system decreased and 100-fold more target gene copies—i.e., 1,700 per μL of template DNA—were needed to achieve a visible PCR product.

Nested PCR achieved a higher sensitivity than direct PCR by detecting as low as 1.7 copies per μL of template DNA in each 20 μL PCR reaction. The experiments to determine the detection limit of the dcpA nested PCR involved two rounds of PCR. First, 10-fold serial dilutions of plasmid DNA containing the partial dcpAB gene of Dhc mccartyi strain KS were used as template in PCR reactions with degenerate primer pair RRF2 (SEQ ID NO: 11) and B1R (SEQ ID NO: 12). The degenerate PCR reactions consisted of (final concentrations): PCR buffer (1×), 3.0 mM of MgCl₂, 0.25 mM of each deoxynucleoside triphosphate (ABI), primers RRF2 (SEQ ID NO: 11) and B1R (SEQ ID NO: 12) (0.5 μM each), 2 μL of serially diluted plasmid (1.7×10⁸-1.7×10⁰ per μL of template DNA), and 2.5 U of AmpliTaq polymerase (ABI). The following thermocycler temperature program for the degenerate PCR was used: 94° C. for 2 min, 10 seconds (1 cycle); 94° C. for 30 seconds, 48.0° C. for 45 seconds, and 72° C. for 2 min, 10 seconds (30 cycles); and 72° C. for 6 min. Following PCR, 2 μL of the resulting degenerate PCR product was used as template in a second PCR reaction with the dcpA-specific primers dcpA-360F (SEQ ID NO: 17)/dcpA-1449R (SEQ ID NO: 16). For all PCR assays, five μL of PCR product were mixed with 1 μL of 6× loading dye and resolved in 1% (wt/vol) agarose gels and stained with ethidium bromide (1 μg/mL). The detection limit was determined as the lowest dilution that yielded a visible amplicon band in an ethidium bromide stained gel. As discussed above, for this nested PCR, 1.7 gene copies per μL of template DNA were needed to produce amplicons that generated a visible band on the agarose gel.

### Example 8

### Probes and Primers

Some of the probes and primers disclosed herein and/or useful in the disclosed invention are set forth in Table 2:

TABLE 2
Probes and Primers

| Probe/Primer | | |
|---|---|---|
| Name | Sequence | SEQ ID NO |
| RRF2 | SHMGBMGWGATTTYATGAARR | SEQ ID NO: 11 |
| | where R = A/G; M = A/C; S = C/G; W = A/T; Y = C/T; | |
| | B = C/G/T; H = A/C/T | |
| B1R | CHADHAGCCAYTCRTACCA | SEQ ID NO: 12 |
| | where R = A/G; Y = C/T; D = A/G/T; H = A/C/T | |
| M13F | GTAAAACGACGGCCAGT | SEQ ID NO: 13 |
| M13R | GGAAACAGCTATGACCATG | SEQ ID NO: 14 |
| dcp_up120F | GCTCCTGGCAGAGCCGTCAGT | SEQ ID NO: 15 |
| dcpA-1449R | TTTAAACAGCGGGCAGGTACTGGT | SEQ ID NO: 16 |
| dcpA-360F | TTGCGTGATCAAATTGGAGCCTGG | SEQ ID NO: 17 |
| dcpA-1257F | CGATGTGCCAGCCATTGTGTCTTT | SEQ ID NO: 18 |
| Dhc 1200F | CTGGAGCTAATCCCCAAAGCT | SEQ ID NO: 19 |
| dcpA- | ACGTCATCTCAGATGAAGGCAGAGCT | SEQ ID NO: 20 |
| 1426Probe | | |
| DhcRC 2000F | TGAGTCGCCGAGATTTTATGAA | SEQ ID NO: 21 |
| DhcRC 2000R | TGCACCCATGCTAGCTATGG | SEQ ID NO: 22 |
| DhcRC | TCCATAGGTTTAGGTTCCGC | SEQ ID NO: 23 |
| 2000Probe | | |
| DhcRC 2100F | CGGCCCTACGCCATCA | SEQ ID NO: 24 |
| DhcRC 2100R | GCAGCAGTCTCTTCTTTGGTAACA | SEQ ID NO: 25 |
| DhcRC | CTGGAACATTAGCCTCAAT | SEQ ID NO: 26 |
| 2100Probe | | |
| DhcRC 2200F | GCCAGGTTTTACATTGCGTGAT | SEQ ID NO: 27 |
| DhcRC 2200R | TCCCGTTTGTCCTCTATCTAACG | SEQ ID NO: 28 |
| DhcRC | AATTGGAGCCTGGGCC | SEQ ID NO: 29 |
| 2200Probe | | |
| DhcRC 2300F | CCGCCTGAAGGTTTTCGA | SEQ ID NO: 30 |
| DhcRC 2300R | GCCTTCCCACTTCGGTACAC | SEQ ID NO: 31 |
| DhcRC | TAAAGTCACCCATGAAAC | SEQ ID NO: 32 |
| 2300Probe | | |
| DhcRC 2400F | AAGGCTCCGAAACGGAAAA | SEQ ID NO: 33 |
| DhcRC 2400R | CGCCCCTGCACCAAATT | SEQ ID NO: 34 |
| DhcRC | ATTCATGATTCGAACTTTCCTGA | SEQ ID NO: 35 |
| 2400Probe | | |
| DhcKS 2500F | CCAGGTTTTACATTGCGTGATC | SEQ ID NO: 36 |
| DhcKS 2500R | TCCCGTTTGTCCTCTATCTAACG | SEQ ID NO: 37 |
| DhcKS | AATTGGAGCCTGGGCC | SEQ ID NO: 38 |
| 2500Probe | | |
| DhcKS 2600F | CCGCCTGAAGGTTTTCGA | SEQ ID NO: 39 |
| DhcKS 2600R | CTTCCCACTTCGGTACACCTAAA | SEQ ID NO: 40 |
| DhcKS | AATTAAAGTCACCCATGAAA | SEQ ID NO: 41 |
| 2600Probe | | |
| DhcKS 2700F | TGAGTCGCCGAGATTTTATGAA | SEQ ID NO: 42 |
| DhcKS 2700R | TGCACCCATGCTAGCTATGG | SEQ ID NO: 43 |
| DhcKS | TCCATAGGTTTAGGTTCCGC | SEQ ID NO: 44 |
| 2700Probe | | |
| DhcKS 2800F | CCGGCCCTACGCCATT | SEQ ID NO: 45 |
| DhcKS 2800R | GCAGCAGTCTCTTCTTTGGTAACA | SEQ ID NO: 46 |
| DhcKS | CTGGAACATTAGCCTCAAT | SEQ ID NO: 47 |
| 2800Probe | | |
| DhcKS 2900F | AAGGCTCCGAAACGGAAAA | SEQ ID NO: 48 |
| DhcKS 2900R | CGCCCCTGCACCAAATT | SEQ ID NO: 49 |
| DhcKS | ATTCATGATTCGAACTTTCCTGA | SEQ ID NO: 50 |
| 2900Probe | | |
| Bac8F | AGAGTTTGATCCTGGCTCAG | SEQ ID NO: 51 |
| Bac1541R | AAGGAGGTGATCCAGCCGCA | SEQ ID NO: 52 |
| Dhc 1271R | CAACTTCATGCAGGCGGG | SEQ ID NO: 53 |
| Dhc 1240Probe | TCCTCAGTTCGGATTGCAGGCTGAA | SEQ ID NO: 54 |
| dcpA491qF | GAAGGCTCCGAAACGGAAA | SEQ ID NO: 55 |
| dcpA549qR | TTCCTGAGGCAATTTGGTGC | SEQ ID NO: 56 |
| dcpA511qP- | CGCATTCATGATTCGAA | SEQ ID NO: 57 |
| MGB | | |

Primers that can be used to amplify or detect the dcpA or dcpB gene or portions thereof or bacteria that include the dcpA or dcpB gene include, for example, RRF2 (SEQ ID NO: 11) and B1R (SEQ ID NO: 12), dcp_up120F (SEQ ID NO: 15) and dcpA-1449R (SEQ ID NO: 16); dcpA-360F (SEQ ID NO: 17) and dcpA-1449R (SEQ ID NO: 16); dcpA-1257F (SEQ ID NO: 18) and dcpA-1449R (SEQ ID NO: 16); DhcRC 2000F (SEQ ID NO: 21) and DhcRC 2000R (SEQ ID NO: 22); DhcRC 2100F (SEQ ID NO: 24) and DhcRC 2100R (SEQ ID NO: 25); DhcRC 2200F (SEQ ID NO: 27) and DhcRC 2200R (SEQ ID NO: 28); DhcRC 2300F (SEQ ID NO: 30) and DhcRC 2300R (SEQ ID NO: 31); DhcRC 2400F (SEQ ID NO: 33) and DhcRC 2400R (SEQ ID NO: 34); DhcRC 2500F (SEQ ID NO: 36) and DhcRC 2500R (SEQ ID NO: 37); DhcRC 2600F (SEQ ID NO: 39) and DhcRC 2600R (SEQ ID NO: 40); DhcRC 2700F (SEQ ID NO: 42) and DhcRC 2700R (SEQ ID NO: 43); DhcRC 2800F (SEQ ID NO: 45) and DhcRC 2800R (SEQ ID NO: 46); DhcRC 2900F (SEQ ID NO: 48) and DhcRC 2900R (SEQ ID NO: 49); and dcpA491qF (5′-GAAGGCTCCGAAACGGAAA-3′; SEQ ID NO: 55) and dcpA549qR (5′-TTCCTGAGGCAATTTGGTGC-3′; SEQ ID NO: 56).

Primers and probes that can be used in Quantitative Real-Time PCR (qPCR) assays to, for example, amplify, detect or quantitate the dcpA gene or portions thereof or bacteria that include the dcpA gene include, for example:
dcpA-1257F (SEQ ID NO: 18), dcpA-1449R (SEQ ID NO: 16), and dcpA-1426Probe (SEQ ID NO: 20);
DhcRC 2000F (SEQ ID NO: 21), DhcRC 2000R (SEQ ID NO: 22), and DhcRC 2000Probe (SEQ ID NO: 23);
DhcRC 2100F (SEQ ID NO: 24), DhcRC 2100R (SEQ ID NO: 25), and DhcRC 2100Probe (SEQ ID NO: 26);
DhcRC 2200F (SEQ ID NO: 27), DhcRC 2200R (SEQ ID NO: 28), and DhcRC 2200Probe (SEQ ID NO: 29);
DhcRC 2300F (SEQ ID NO: 30), DhcRC 2300R (SEQ ID NO: 31), and DhcRC 2300Probe (SEQ ID NO: 32);
DhcRC 2400F (SEQ ID NO: 33), DhcRC 2400R (SEQ ID NO: 34), and DhcRC 2400Probe (SEQ ID NO: 35);
DhcRC 2500F (SEQ ID NO: 36), DhcRC 2500R (SEQ ID NO: 37), and DhcRC 2500Probe (SEQ ID NO: 38);
DhcRC 2600F (SEQ ID NO: 39), DhcRC 2600R (SEQ ID NO: 40), and DhcRC 2600Probe (SEQ ID NO: 41);
DhcRC 2700F (SEQ ID NO: 42), DhcRC 2700R (SEQ ID NO: 43), DhcRC 2700Probe (SEQ ID NO: 44);
DhcRC 2800F (SEQ ID NO: 45), DhcRC 2800R (SEQ ID NO: 46), and DhcRC 2800Probe (SEQ ID NO: 47);
DhcRC 2900F (SEQ ID NO: 48), DhcRC 2900R (SEQ ID NO: 49), and DhcRC 2900Probe (SEQ ID NO: 50); and
dcpA491qF (5′-GAAGGCTCCGAAACGGAAA-3′; SEQ ID NO: 55), dcpA549qR (5′-TTCCTGAGGCAATTTGGTGC-3′; SEQ ID NO: 56) and dcpA511qP-MGB (5′-6FAM-CGCATTCATGATTCGAA-MGB-3′; SEQ ID NO: 57).

### Example 9

### dcpA Gene Copy Number and Transcriptional Analysis

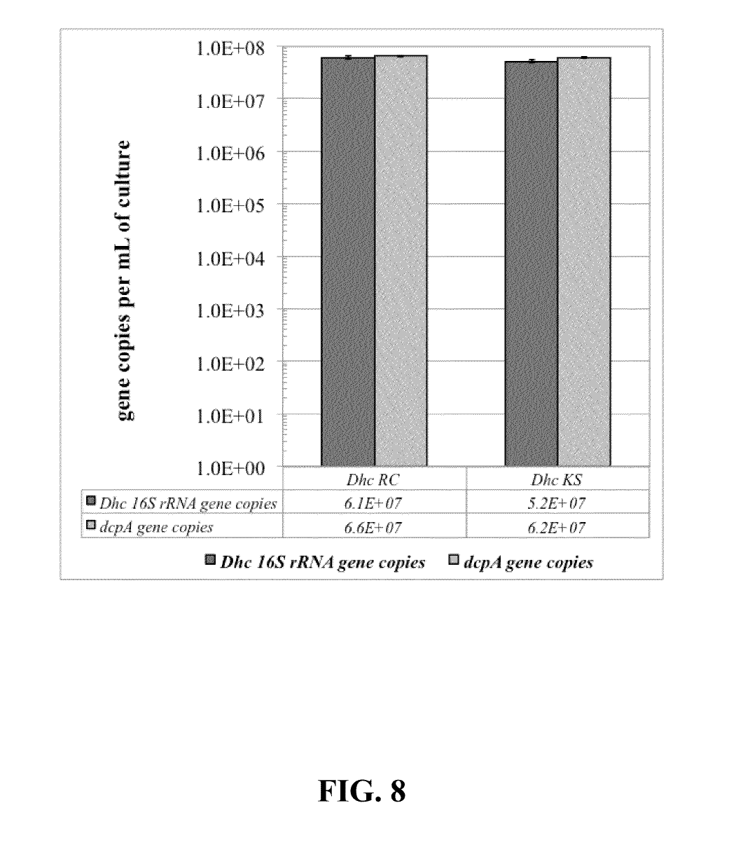

dcpA gene copy numbers and transcription levels were quantified by qPCR and reverse transcription- (RT-) Real-Time PCR. The assay conditions for the reactions targeting Dhc 16S rRNA, rpoB transcripts and luciferase transcripts were performed as described previously (He, J., et al. (2003) Appl. Environ. Microbiol. 69:996-1003, Fung, J. M., et al. (2007) Appl. Environ. Microbiol. 73:4439-45, Johnson, D. R., et al. (2005) Appl. Environ. Microbiol. 71:3866-3871). Quantitative Real-Time PCR using genomic DNA from Dhc mccartyi strain RC and strain KS cultures as template DNA was performed in order to quantify the dcpA gene copies per mL of culture. For this, 1 mL of culture suspension was centrifuged at 16,000 g for 5 min. The supernatant was decanted and the resulting pellets stored at −20° C. The DNeasy Blood and Tissue Kit (Qiagen, Valencia, Calif., USA) was used according to the manufacturer's protocol with the following modifications: 10 μL of achromopeptidase (25 mg/mL), 45 μL of proteinase K (25 mg/mL), and 20 μL of lysozyme (100 mg/mL) were added to improve cell lysis and DNA recovery. DNA was eluted from a column with 100 μL of Elution Buffer provided with the DNeasy Blood and Tissue Kit, and 2 μL of this DNA was used as template in 20 μL qPCR reactions. The dcpA TaqMan assays included 1×TaqMan Universal PCR master mix (ABI; product no. 4304437), 300 nM of probe, 300 nM each of reverse and forward primers and 2 μL of purified DNA as template in a total volume of 20 μL. The PCR cycle parameters were as follows: 2 min at 50° C. (to activate the AmpErase UNG present in the master mix used to minimize the contamination of previous reaction products), denaturation for 10 min at 95° C., followed by 40 cycles of 15 seconds at 95° C. and 1 min at 60° C. For the quantification of the dcpA gene, the dcpA-1257F (SEQ ID NO: 18) and dcpA-1449R (SEQ ID NO: 16) primers and the dcpA-1426Probe (SEQ ID NO: 20) probe was used. For the quantification of Dhc, the group-specific 16S rRNA gene-targeted primers Dhc 1200F (5′-CTGGAGCTAATCCCCAAAGCT-3′; SEQ ID NO: 19) and Dhc 1271R (5′-CAACTTCATGCAGGCGGG-3′; SEQ ID NO: 53) and the probe Dhc-1240Probe (5-6 FAM-TCCTCAGTTCGGATTGCAGGCTGAA-3′-TAMPA; SEQ ID NO: 54) were used as described in He, J., et al. (2003) Appl. Environ. Microbiol. 69:996-1003. The known Dhc genomes possess one 16S rRNA gene copy, and the 16S rRNA gene copy number equals the Dhc cell number. The number of dcpA genes per Dhc genome in this experiment was calculated by taking the number of dcpA gene copies detected per mL of culture and dividing it by the total number of Dhc 16S rRNA genes detected per mL of culture. The results from this experiment, which are shown graphically in FIG. 8, demonstrate that there is one copy of the dcpA gene in the Dhc genome.

For the transcriptional analysis, qPCR used cDNA templates generated from active 1,2-dichloropropane dechlorinating cultures. Culture growth conditions, RNA extraction and cDNA synthesis were performed as previously described herein. All RT-Real-Time PCR data were corrected for % loss (or % recovery) as described previously (Johnson, D. R., et al. (2005) Appl. Environ. Microbiol. 71:3866-3871; Ritalahti, K. M., et al. (2010) In K. N. Timmis (ed.), Handbook of Hydrocarbon and Lipid Microbiology. Springer Berlin Heidelberg 32:3671-3685) by using the luciferase transcripts as an internal standard. rpoB transcripts were quantified as an indicator of metabolic activity. This housekeeping gene is conserved in Dhc genomes and it has been successfully used for normalization in Dhc transcriptional studies (Rahm, B. G., et al. (2006) Appl. Environ Microbiol. 72:5486-5491, Fung, J. M., et al. (2007) Appl. Environ. Microbiol. 73:4439-45, Chow, W. L., et al. (2010) ISME J. 4:1020-30). When applicable, dcpA transcript abundances were normalized to rpoB or to dcpA gene copy numbers. Since previous results showed that the dcpA gene exists in one copy in the RC and KS genome, dcpA gene copy numbers and Dhc cell numbers can be used interchangeably. Starved cultures (i.e., cultures that had consumed all 1,2-dichloropropane for at least 1 month) served as negative controls.

The dcpA TaqMan assays consisted of 1×TaqMan Universal PCR master mix (ABI; product no. 4304437), 300 nM of probe, 300 nM each of reverse and forward primers and 2 μL of purified DNA and/or diluted cDNA (1:50 and 1:100) as template in a total volume of 20 μL. The PCR cycle parameters were as follows: 2 min at 50° C. (to activate the AmpErase UNG present in the master mix used to minimize the contamination of previous reaction products), denaturation for 10 min at 95° C., followed by 40 cycles of 15 seconds at 95° C. and 1 min at 60° C. For the quantification of the dcpA gene, the dcpA-1257F (SEQ ID NO: 18) and dcpA-1449R (SEQ ID NO: 16) primers and the dcpA-1426Probe (SEQ ID NO: 20) probe was used.

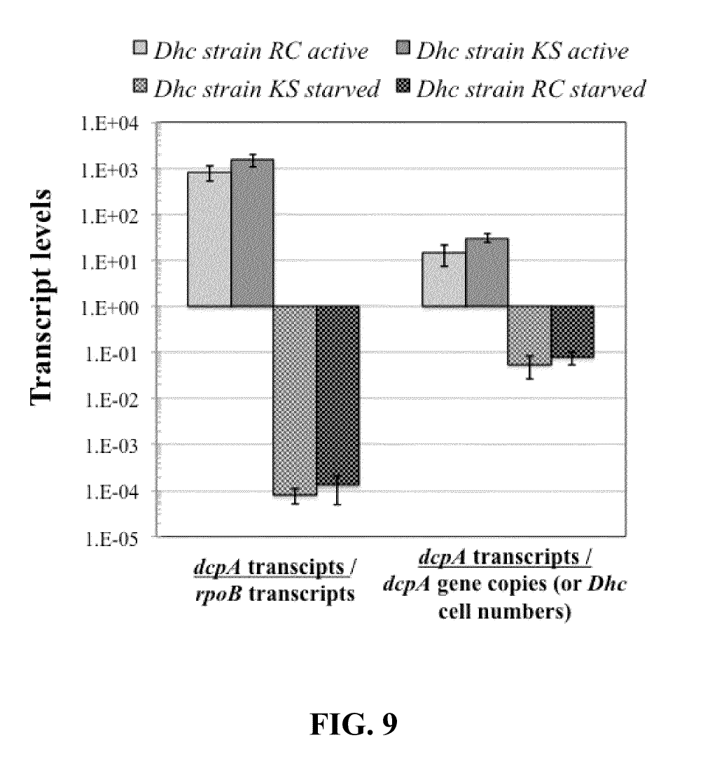

Gene copies per reaction or per μL of template DNA in standards or unknown samples were calculated as described previously (Ritalahti, K. M., et al. (2006) Appl. Environ. Microbiol. 72:2765-2774). The numbers of target genes were normalized to milliliters of culture. The Dhc, rpoB and luciferase qPCR assays were performed as described previously (He, J., et al. (2003) Appl. Environ. Microbiol. 69:996-1003, Fung, J. M., et al. (2007) Appl. Environ. Microbiol. 73:4439-45, Johnson, D. R., et al. (2005) Appl. Environ. Microbiol. 71:3866-3871). Samples were run in triplicate in the ABI 7500 Fast Quantitative Real-Time PCR (Applied Biosystems) and final values represent the average of at least three biological replicate cultures. The results from this experiment, which are shown graphically in FIG. 9, demonstrate that the transcript levels of the dcpA gene are increased in Dhc strains that are exposed to 1,2-dichloropropane as compared to Dhc strains that had not consumed 1,2-dichloropropane for at least 1 month.

### Example 10

### Detection of dcpA in Microcosms

Microcosm Cultures.

For microcosm set-up, sediments were collected in sterile Mason jars and/or plastic tubes from Third Creek, Knoxyille, Tenn. (3 different locations), Neckar River and an agricultural soil near Stuttgart, Germany (Trester sample). Samples from contaminated aquifers were also collected and included samples from Barra Mansa (Brazil; 2 samples), Fort Pierce (Florida, USA; 5 samples) and Waynesboro (Georgia). All samples were delivered to the laboratory by overnight carrier and stored at 4° C. for no more than one month, or immediately processed. Additional information about these samples, including the sample location, the sample type, the major reported contaminants at the sample location, the date the sample was collected, and whether propene or the dcpA gene were detected in the sample, is provided in Table 3.

TABLE 3
Characteristics of site materials and results of microcosm amended with 1,2-dichloropropane

| | | | Major Reported | | Dechlorination end | |
|---|---|---|---|---|---|---|
| Sample designation | Sample location | Sample type | contaminants | Date of collection | products | dcpA gene detected |
| TRS1 | Third creek, | sediment | PCE, TCE, 1,1,1-TCA^{a} | February 2011 | propene | + |
| | Knoxville, TN, | | | | | |
| | USA | | | | | |
| TRS2 | Third creek, | sediment | PCE, TCE, 1,1,1-TCA^{a} | February 2011 | propene | + |
| | Knoxville, TN, | | | | | |
| | USA | | | | | |
| TRS3 | Third creek, | sediment | PCE, TCE, 1,1,1-TCA^{a} | March 2011 | propene | + |
| | Knoxville, TN, | | | | | |
| | USA | | | | | |
| Neckar | Neckar River | sediment | None^{b} | May 2011 | propene | + |
| | Stuttgart, | | | | | |
| | Germany | | | | | |
| Trester | Stuttgart, | sediment | None^{c} | May 2011 | propene | + |
| | Germany | | | | | |
| Brazil 001-ST-SO | Barra Mansa, | sediment | Chloroform, CCl₄^{d} | August 2010 | —^{e} | + |
| 2.70-2.94 m | Brazil | | | | | |
| Brazil 002-ST-SO | Barra Mansa, | sediment | Chloroform, CCl₄^{d} | August 2010 | —^{e} | + |
| 5.70-5.85 m | Brazil | | | | | |
| Waynesboro | Waynesboro, | groundwater | 1,2-D and 1,2- | August 2010 | — | − |
| MW13D-12J811 | Burke County, | | dichloroethane | | | |
| | GA, USA | | | | | |
| Ft Pierce 1-MW46 | Ft. Pierce, | sediment | 1,2-D, but outside the plume | August 2010 | — | − |
| 22.5-26.5 m | St. Lucie County, | | area | | | |
| | FL, USA | | | | | |
| Ft Pierce 2-MW49 | Ft. Pierce, | sediment | Same as above | August 2010 | — | − |
| 26-27 m | St. Lucie County, | | | | | |
| | FL, USA | | | | | |
| Ft Pierce 3- | Ft. Pierce, | sediment | Same as above | August 2010 | — | − |
| MW4946-47 m | St. Lucie County, | | | | | |
| | FL, USA | | | | | |
| Ft Pierce 4-MW47 | Ft. Pierce, | sediment | Same as above | August 2010 | — | − |
| 47-48 m | St. Lucie County, | | | | | |
| | FL, USA | | | | | |
| Ft Pierce 5-MW49 | Ft. Pierce, | sediment | Same as above | August 2010 | — | − |
| 95-98 m | St. Lucie County, | | | | | |
| | FL, USA | | | | | |

—; no dechlorination after 90 days incubation
^{a}site has a history of PCE (perchloroethylene), TCE (trichloroethylene), and 1,1,1-TCA (1,1,1-trichloroethane) contamination
^{b}site has no history of previous chlorinated solvents contamination but is near urban/industrial areas
^{c}site has been impacted for many years with residues from wine making processes
^{d}site contains up to 7.760 ppb of chloroform and 125 μg/L of carbon tetrachloride (CCl₄); chlorinated methanes are known inhibitors of dechlorination
^{e}small amounts of 1-CP (1-chlorophenol) or 2-CP (2-chlorophenol) were detected in live microcosm but also in negative controls
^{f}Ft. Pierce is contaminated with 1,2-dichloropropane (1,2-D) (up to 30,000 ppb) but the sediment tested here were for wells outside the plume area

Microcosms were established as previously described (He, J., (2002) Environ. Sci. Technol. 36:3945-3952) with the following modifications: sterile 60 mL glass serum bottles (nominal capacity) were used to establish duplicate microcosms and 40 mL of reduced mineral salts medium (Löffler, F. E et al. (1996). Appl. Environ. Microbiol. 62:3809-3813) were used, except that 5 mM lactate (instead of acetate) and a concentration of 0.2 mM (aqueous concentration) of 1,2-dichloropropane were used. Each serum bottle received approximately 2 g of solids, 3% H₂-97% N₂ (vol/vol) from the anoxic chamber. Microcosms established with groundwater collected from the Waynesboro site were set-up using 20 mL of groundwater plus 20 mL of reduced mineral salts medium (Löffler, F. E et al. (1996). Appl. Environ. Microbiol. 62:3809-3813). Microcosms that received autoclaved sediment or groundwater served as negative controls. All microcosms and enrichments were incubated statically in the dark at room temperature. Propene and 1,2-dichloropropane concentrations were monitored by manually injecting 0.1 mL headspace samples into the GC-FID. After all 1,2-dichlroropropane was dechlorinated to propene, 3% inocula (vol/vol) were sequentially transferred to culture vessels with fresh medium. Prior to isolating DNA from the microcosm cultures (as described below), the eleven microcosm samples were amended with 1,2-dichloropropane.

DNA Isolation.

DNA isolation from environmental samples and microcosms used the MoBio Power Soil DNA kit (MoBio Laboratories Inc., Carlsbad, Calif., USA) following the manufacturer's protocol. To improve cell lysis, the samples were incubated for 10 minutes at 70° C. before the bead beating step. The Omni-Bead Ruptor 24 (OMNI International Inc., Kennesaw, Ga. USA) was used in the homogenization and bead-beating step for sediment samples. To isolate DNA from groundwater samples, 500 mL were filtered through a MoBio polyethersulfone filter membrane with a 0.22 μm pore size (MoBio Laboratories Inc., Carlsbad, Calif., USA, part number 14880-50-WF) and transferred (with sterile forceps) to the bead tube provided with the PowerWater DNA Bead Tube Isolation Kit (MoBio Laboratories Inc., Carlsbad, Calif., USA). Extractions from groundwater samples were performed following the manufactures recommendations and, to aid in cell lysis, the filter inside the Bead Tube was incubated at 65° C. for 10 minutes before bead beating in the vortexer. For environmental samples, DNA from 2 to 4 different extractions were pooled and combined to give a single DNA extract per site, always keeping track of the mass or volume they represented. The purified DNA was quantified using a Nanodrop spectrophotometer (Thermo Fisher Scientific, Wilmington, Del., USA) and stored at −20 or −80 degrees Celsius.

Analysis of Microcosm Culture DNA.

DNA isolated from the eleven microcosm samples were analyzed for the presence of the dcpA gene. Nested PCR assays were performed by a first amplification round using 2 μL of template DNA and the degenerate primers RRF2 (SEQ ID NO: 11) and B1R (SEQ ID NO: 12) as previously described (Krajmalnik-Brown, R., et al., (2004) Appl. Environ. Microbiol. 70: 6347-6351). Subsequently, a second round of PCR using the dcpA-specific primers dcpA-360F (5′-TTGCGTGATCAAATTGGAGCCTGG-3′; SEQ ID NO: 17) and dcpA-1449R (5′-TTTAAACAGCGGGCAGGTACTGGT-3′; SEQ ID NO: 16) was performed. The dcpA gene was detected in seven of the eleven samples: the 3 Third Creek samples (Knoxyille, Tenn.), the Neckar River sample (Stuttgart, Germany), the Trester sample (Stuttgart, Germany), and the 2 Barra Mansa samples (Brazil) (see FIG. 10). Out of the seven sediments where the dcpA gene was detected, five showed dechlorination of 1,2-dichloropropane to propene: the three Third Creek samples (Knoxyille, Tenn.), the Neckar River sample (Stuttgart, Germany), and the Trester sample (Stuttgart, Germany) (see FIG. 10). These results show a correlation of 1,2-dichloropropane dechlorination and propene production in microcosms where the dcpA gene was detected. Interestingly, the samples where the dcpA gene was detected (and that exhibited dechlorination activity) represent freshwater sediments from both contaminated and pristine sites. For example, the Third Creek, Knoxyille, Tenn. locations (3 different locations) are within an area that has a history of exposure to chlorinated solvents. However, the samples from Germany (Trester and Neckar) are not known to have been previously impacted by chlorinated solvents. The only microcosm samples where the dcpA gene was detected but no 1,2-dichloropropane dechlorinating activity was detected are from a contaminated site in the industrial town of Barra Mansa in Brazil. This site is contaminated with other chloroorganic contaminants including carbon tetrachloride, a known inhibitor of microbial activity. In most cases not even microbial production of methane was observed in this microcosm, which is further evidence of toxicity and inhibition to the microbial community.

### Example 11

### Detection of dcpA in Other Environmental Samples

Additional sources of DNA were screened for the dcpA gene. DNA samples representative of wells [MW-2S (22.5 ft), MW-20 (72.5 ft), MW-26 (47.5 ft), and MW61 (72.5 ft)] from the source zone (plume) in Fort Pierce (Florida, USA) were kindly provided by Microbial Insights. These DNA samples were extracted from carbon activated beads (Bio-Sep® beads) inside of Bio-Traps® that were deployed into the monitoring wells. The beads absorb electron donor and acceptors present in the groundwater and are colonized by microbial communities. DNA samples from the uranium-contaminated U.S. Department of Energy Integrated Field-Scale Subsurface Research Challenge (IFC) site near Oak Ridge, Tenn. were also obtained. In addition, the DNA extracted from groundwater samples of Area 3 taken during a successful field pilot study for uranium bio-reduction (Wu, W., et al. (2006) Environ. Sci. Technol. 40:3978-3985) were kindly provided by Youlboong Sung. These samples corresponded to wells FW104, FW103, FW100-2, and FW100-3 within the ORNL pilot study that encompassed a well recirculation system where inner wells received electron donor and wells from the outer loop served for hydrological protection (see Amos, B. K., et al. (2007) Appl. Environ. Microbiol. 73:6898-904). Additional information about these environmental samples, including the sample location, the DNA source used for analyzing the sample, the date the sample was collected, a description of the well, the concentration of 1,2-dichloropropane in the sample, the depth of the well, and whether the dcpA gene or propene were detected in the sample, is provided in Table 4.

TABLE 4
Characteristics of Environmental Samples

| | | | | | 1,2-DCP | | | |
|---|---|---|---|---|---|---|---|---|
| Sample | | | Date of | | concentration | | dcpA gene | Propene |
| designation | Sample location | DNA source | collection | Well Description | μg/L | Depth | detected | detected |
| MW-2S | Ft. Pierce, St. Lucie County, | Biobead | July, 2011 | Inside sourcezone | 14000 | 22.5 ft | − | no |
| | FL, USA | | | | | | | |
| MW-20 | Ft. Pierce, St. Lucie County, | Biobead | February 2011 | Inside sourcezone | 810 | 72.5 ft | + | yes |
| | FL, USA | | | | | | | |
| MW-26 | Ft. Pierce, St. Lucie County, | Biobead | March 2011 | Inside sourcezone | 17000 | 47.5 ft | + | yes |
| | FL, USA | | | | | | | |
| MW-61 | Ft. Pierce, St. Lucie County, | Biobead | May, 2011 | Inside sourcezone | 140 | 72.5 ft | + | yes |
| | FL, USA | | | | | | | |
| FW 100-2 | IFC site, Oakridge, TN | GW | February 2004 | Multilevel sample well in | nt | 40.0 ft | + | nt |
| | | | | outer loop | | | | |
| FW 100-3 | IFC site, Oakridge, TN | GW | February 2004 | Multilevel sample well in | nt | 35.0 m | + | nt |
| | | | | outer loop | | | | |
| FW 102-2 | IFC site, Oakridge, TN | GW | August 2005 | Multilevel sample well in | nt | 40.0 ft | − | nt |
| | | | | inner loop | | | | |
| FW 103 | IFC site, Oakridge, TN | GW | February 2004 | Outer loop extraction | nt | n/a | + | nt |
| | | | | well | | | | |

GW = groundwater
Nt = not tested
1,2-DCP = 1,2-dichloropropane

DNA from the environmental samples was prepared as described above in Example 10 and subjected to a nested PCR approach. A first amplification round used 2 μL of template DNA and the degenerate primers RRF2 (SEQ ID NO: 11) and B1R (SEQ ID NO: 12) as previously described (Krajmalnik-Brown, R., et al. (2004) Appl. Environ. Microbiol. 70:6347-6351). Subsequently, a second round of PCR using the dcpA-specific primers dcpA-360F (5′-TTGCGTGATCAAATTGGAGCCTGG-3′; SEQ ID NO: 17) and dcpA-1449R (5′ TTTAAACAGCGGGCAGGTACTGGT-3′; SEQ ID NO: 16) was performed. The expected amplicon size was 1,089 bp. The depA PCR reactions consisted of (final concentrations) PCR buffer (1×), 2.5 mM of MgCl₂, 250 μM of each deoxynucleoside triphosphate (ABI), primers (250 nM each), and 2.5 U of AmpliTaq polymerase (ABI). The following thermocycler temperature program for the amplification of the dcpA gene was used: 94° C. for 2 min 10 seconds (1 cycle); 94° C. for 30 seconds, 56° C. for 45 seconds, and 72° C. for 2 min 10 seconds (30 cycles); and 72° C. for 6 min. Five μL of PCR product mixed with 1 μL of 6× loading dye were resolved on 1% (wt/vol) agarose gels and stained with ethidium bromide (1 μg/mL). All dcpA amplicons were cloned and sequenced.

Three out of the four DNA samples representative of wells from the source zone (plume) in Fort Pierce (Florida, USA) yielded positive results for the dcpA gene: MW-20, MW-26 and MW-61 (see FIG. 11). The presence of the dcpA gene in the DNA from these samples correlated with the detection of propene in these three wells. While the dcpA gene was not detected in the MW-25 sample, there was also no propene detected in this sample (see FIG. 11). Three additional samples from the mixed-waste contaminated site in Oak Ridge, Tenn.—FW103 (outer loop extraction well), FW100-2 (multilevel sample well in outer loop), and FW 100-3 (multilevel sample well in the outer loop)—showed an amplicon of the expected size for dcpA, and only one sample from FW 102-2 (multilevel sample well in inner loop) was negative (see FIG. 11) indicating that the dcpA gene copy numbers in this sample were absent or below the detection limit.

### Example 12

### DcpA Identification by Enzymatic Assays, Polyacrylamide Gel Electrophoresis and 2D-LC-MS/MS

A combination of protein techniques such as Blue Native Polyacrylamide Gel Electrophoresis (BN-PAGE), in vitro enzyme assays, SDS-PAGE, and proteomic analysis showed that cells of Dehalogenimonas lykanthroporepellens strain BL-DC-9 grown in 1,2-dichloropropane express a DcpA protein.

The Dehalogenimonas lykanthroporepellens strain BL-DC-9 was kindly provided by Drs. Jun Yan and William Moe. Two liters of Dehalogenimonas lykanthroporepellens strain BL-DC-9 were grown as previously described (Moe, W. M., et al. (2009). Int. J. Syst. Evol. Microbiol. 59:2692-2697) with 5 mM sodium acetate, vitamins (Wolin, E. A., et al. (1963) J. Biol. Chem. 238:2882-2886), 5-10 mL of hydrogen and 20-30 mg/L 1,2-dichloropropane. Dechlorination was measured by GC-FID and cultures received a second feeding of 1,2-dichloropropane after cultures had dechlorinated all 1,2-dichloropropane to propene. Cells were collected by centrifugation and subjected to lysis by adding a small amount of spherical lead-free soda lime glass bacteria disrupter beads (0.1 mm, VWR cat no. 101454-154) and vortexing at maximum speed using a desktop vortex unit (Vortex Genie 2, Scientific Industries, Inc., Bohemia, N.Y.) for 10 minutes. Samples were kept chilled by placing them periodically on ice every couple of minutes during the vortexing procedure. Cell-free extracts were run on Blue Native (BN) Polyacrylamide Gel Electrophoresis. Coomassie stain was used to visualize the most abundant proteins in the sample and additional lanes were run in parallel for the SDS-PAGE and enzyme activity assays as described by Tang et al., “Functional Characterization of Dehalococcoides Reductive Dehalogenases Using Blue Native Polyacrylamide Gel Electrophoresis,” (2012) Appl. & Environ. Microbiol. (submitted). In summary, by using the stained lane as a guide, bands were excised from the gel followed by an in vitro enzyme activity assay to identify the protein fraction with dehalogenase activity. The excised gel segments were placed in 2 mL vials containing a mixture of 4 mM methyl viologen, 4 mM titanium III citrate, 0.5 mM 1,2-dichloropropane and 100 mM Tris, pH 7.4. Vials were stirred for 18 hours inside an anoxic chamber. For a negative control, the same mixture described above was placed in a vial without a gel segment. Headspace samples (0.1-0.2 mL) from the vials were injected into the GC-FID as described previously (Amos B. K., et al. (2007) Environ. Sci. Technol. 41:963-970) except that a GC inlet split ratio of 1:1 was used. A band around 50 kD showed 1,2-dichloropropane-to-propene dechlorination activity. From the additional lanes run in parallel for the SDS-PAGE, the corresponding gel band was placed in SDS elution buffer (100 mM Tris, pH 7.0; 0.1% SDS) and incubated at 4° C. overnight. The SDS elution buffer containing the gel piece was concentrated by loading it onto a low-binding 10 kDa Microcon ultrafiltration unit (Millipore, Billerica, Mass., USA) following the manufacturer's recommendations. Concentrated samples were run on SDS-PAGE using Coomassie stain to visualize the bands (as explained in Tang et al., “Functional Characterization of Dehalococcoides Reductive Dehalogenases Using Blue Native Polyacrylamide Gel Electrophoresis,” (2012) Appl. & Environ. Microbiol. (submitted)) and subsequently, the bands were excised from the gel for LC-MS/MS analysis. Coomassie stained gel bands were excised from the SDS-PAGE gel and rinsed in HPLC grade water. In-gel digestion of proteins was performed as described previously (Shevchenko, A. et al. (2006) Nature Protocols. 1:2856-60). Briefly, gel pieces were cut into small pieces and destained for 30 min in 100 mM ammonium bicarbonate/acetonitrile (1:1, vol/vol) at room temperature along with intermittent vortexing. Gel-enmeshed proteins were further subjected to reduction, alkylation and overnight trypsin digestion at 37° C. as described previously (Shevchenko, A. et al. (2006) Nature Protocols. 1:2856-60), and peptides were extracted in 100 μL of extraction buffer (5% formic acid/acetonitrile). The extracted peptide mix (50 μL) was pressure loaded onto an in-house packed biphasic MuDPIT (Multi Dimensional Protein Identification Technology) column packed with ˜3 cm strong cation exchange (SCX) resin (Phenomenex, Torrance, Calif.) and ˜5 cm of reverse phase (RP) C18 resin (Phenomenex, Torrance, Calif.). The sample column was connected to a 15 cm RP packed front column (New Objective, Woburn, Mass.) and analyzed via 2D LC-MS/MS using three salt pulses (30, 60 and 100% of 500 mM ammonium acetate) followed by a 120 min elution gradient (100% Solvent A (95% water, 5% acetonitrile, 0.1% formic acid) to 60% Solvent B (30% water, 70% acetonitrile, 0.1% formic acid)). Peptide fragmentation data were collected using an LTQ or LTQ-orbitrap, operated in data-dependent mode and under the control of the Xcalibur software (Thermo Scientific). The LTQ Orbitrap was set to 30K resolution while the rest of the precincts on either instrument were maintained as described elsewhere (Brown, S. D., et al. (2006) Mol. Cell. Proteomics. 5:1054-1071, Thompson, M. R., et al. (2008) Anal. Chem. 80:9517-9525, Chourey, K., et al. (2010) J. Proteome Res. 9:6615-6622). The MS/MS data obtained were searched against the Dehalogenimonas lykanthroporepellens BL-DC-9 genome (NC_{—}014314.1, downloaded from JGI, April 2012) using the SEQUEST algorithm (Eng, J. K., et al. (1994) J. Am. Soc. Mass Spectrom. 5:976-989). The resultant datasets were sorted using DTASelect (Tabb, D. L., et al. (2002). J. Proteome Res. 1:21-26) set to the following parameters: fully tryptic peptides only with ACN of at least 0.08 and cross-correlation scores (Xcorr) of at least 1.8 (+1), 2.5 (+2), and 3.5 (+3) (Brown, S. D., et al. (2006) Mol. Cell. Proteomics. 5:1054-1071, Thompson, M. R., et al. (2008) Anal. Chem. 80:9517-9525). LC-MS/MS analysis on the protein band with 1,2-dichloropropane dechlorination activity identified the presence of various peptides with homology to the annotated Dehalogenimonas lykanthroporepellens strain BL-DC-9 genome. However, based on spectral abundance, Dehly 0337 (annotated as translation elongation factor) and Dehly_{—}1524 (annotated as reductive dehalogenase) appeared to be prominently represented in the gel slice. Among the proteins present, Dehly_{—}1524 was the only RDase. These data support the results obtained in the Examples described above and confirm that DcpA catalyzes the 1,2-dichloropropane to propene reaction.

All chemicals for proteomic analysis were obtained from Sigma Chemical Co. (St. Louis, Mo.), trypsin was acquired from Promega (Madison, Wis.), formic acid (99%) was obtained from EM Science (Darmstadt, Germany) and HPLC-grade water and acetonitrile from Burdick and Jackson (Muskegon, Mich.). Unless otherwise noted, all temperatures are in degrees Celsius.

Patents, patent applications, publications, product descriptions, and protocols are cited throughout this application, the disclosures of which are incorporated herein by reference in their entireties for all purposes.

1. An isolated polynucleotide having at least 95% sequence identity over the length of the entire reference sequence to a sequence selected from the group consisting of SEQ ID NO: 1, SEQ ID NO: 3, SEQ ID NO: 5, SEQ ID NO: 7, SEQ ID NO: 9, SEQ ID NO: 10, SEQ ID NO: 15, SEQ ID NO: 16, SEQ ID NO: 17, SEQ ID NO: 18, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 55, SEQ ID NO: 56 and SEQ ID NO: 57.

2. The isolated polynucleotide of claim 1, wherein the polynucleotide has at least 99% sequence identity over the length of the entire reference sequence to a sequence selected from the group consisting of SEQ ID NO: 1, SEQ ID NO: 3, SEQ ID NO: 5, SEQ ID NO: 7, SEQ ID NO: 9, SEQ ID NO: 10, SEQ ID NO: 15, SEQ ID NO: 16, SEQ ID NO: 17, SEQ ID NO: 18, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, and SEQ ID NO: 55, SEQ ID NO: 56 and SEQ ID NO: 57.

3. The isolated polynucleotide of claim 1, wherein the polynucleotide comprises a sequence selected from the group consisting of SEQ ID NO: 1, SEQ ID NO: 3, SEQ ID NO: 5, SEQ ID NO: 7, SEQ ID NO: 9, SEQ ID NO: 10, SEQ ID NO: 15, SEQ ID NO: 16, SEQ ID NO: 17, SEQ ID NO: 18, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 55, SEQ ID NO: 56 and SEQ ID NO: 57.

4. The isolated polynucleotide of claim 1, wherein the polynucleotide consists of a sequence selected from the group consisting of SEQ ID NO: 1, SEQ ID NO: 3, SEQ ID NO: 5, SEQ ID NO: 7, SEQ ID NO: 9, SEQ ID NO: 10, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 55, SEQ ID NO: 56 and SEQ ID NO: 57.

5. The isolated polynucleotide of claim 1, wherein the polynucleotide comprises one or more of a reporter dye or a quencher.

6. A recombinant expression vector comprising the polynucleotide of claim 1 operably linked to a regulatory sequence.

7. A cell comprising the recombinant expression vector of claim 6.

8. An organism comprising the recombinant expression vector of claim 6.

9. A vector comprising the polynucleotide of claim 1.

10. The isolated polynucleotide of claim 1, wherein the polynucleotide encodes a reductive dehalogenase enzyme.

11. The isolated polynucleotide of claim 1, wherein the polynucleotide encodes a polypeptide that has at least 95% sequence identity over the length of the entire reference sequence to a sequence selected from the group consisting of SEQ ID NO: 2, SEQ ID NO: 4, SEQ ID NO: 6 and SEQ ID NO: 8.

12. The isolated polynucleotide of claim 1, wherein the polynucleotide encodes a polypeptide that has at least 99% sequence identity over the length of the entire reference sequence to a sequence selected from the group consisting of SEQ ID NO: 2, SEQ ID NO: 4, SEQ ID NO: 6 and SEQ ID NO: 8.

13. A method for identifying a dechlorinating bacterial organism in a sample comprising:
a. contacting the sample with (i) a first oligonucleotide primer comprising a sequence selected from the group consisting of SEQ ID NO: 15, SEQ ID NO: 17, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, and SEQ ID NO: 55; and (ii) a second oligonucleotide primer comprising a sequence selected from the group consisting of SEQ ID NO: 16, SEQ ID NO: 22, SEQ ID NO: 25, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, and SEQ ID NO: 56; and
b. performing PCR on the sample to determine the presence of the dechlorinating bacterial organism in the sample.

14. The method of claim 13, wherein the first oligonucleotide comprises SEQ ID NO: 15 and the second oligonucleotide comprises SEQ ID NO: 16.

15. The method of claim 13, wherein the first oligonucleotide comprises of SEQ ID NO: 55 and the second oligonucleotide comprises SEQ ID NO: 56.

16. A method for quantifying the amount of dechlorinating bacteria present in a sample comprising:
a. contacting the sample with (1) a first oligonucleotide primer comprising a sequence selected from the group consisting of SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, and SEQ ID NO: 55; (ii) a second oligonucleotide primer comprising a sequence selected from the group consisting of SEQ ID NO: 16, SEQ ID NO: 22, SEQ ID NO: 25, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, and SEQ ID NO: 56; and (iii) a probe comprising a sequence selected from the group consisting of SEQ ID NO: 20, SEQ ID NO: 23, SEQ ID NO: 26, SEQ ID NO: 29, SEQ ID NO: 32, SEQ ID NO: 35, SEQ ID NO: 38, SEQ ID NO: 41, SEQ ID NO: 44, SEQ ID NO: 47, SEQ ID NO: 50, and SEQ ID NO: 57; and
b. performing Quantitative Real-Time PCR on the sample to quantify the amount of dechlorinating bacterial present in the sample.

17. The method of claim 16, wherein the first oligonucleotide comprises SEQ ID NO: 18, the second oligonucleotide comprises SEQ ID NO: 16, and the probe comprise SEQ ID NO: 20.

18. The method of claim 16, wherein the first oligonucleotide comprises SEQ ID NO: 55, the second oligonucleotide comprises SEQ ID NO: 56, and the probe comprises SEQ ID NO: 57.

19. The method of claim 16, wherein the first oligonucleotide comprises SEQ ID NO: 21, the second oligonucleotide comprises of SEQ ID NO: 22, and the probe comprises SEQ ID NO: 23.

20. The method of claim 16, wherein the probe comprises one or more of a reporter dye or a quencher.
